SEQUENCE LISTING (21 sequences)

SEQ ID NO: 1 - protein, 14 aa, Helianthus annuus
GRCTKSIPPICFPD

SEQ ID NO: 2 - protein, 14 aa, Artificial Sequence
  Synthetic peptide
GYCSRSYPPVCIPD

SEQ ID NO: 3 - protein, 14 aa, Artificial Sequence
  Synthetic peptide
GICSRSLPPICIPD

SEQ ID NO: 4 - protein, 14 aa, Artificial Sequence
  Synthetic peptide
GVCSRSLPPICWPD

SEQ ID NO: 5 - protein, 14 aa, Artificial Sequence
  Synthetic peptide
GMCSRSYPPVCIPD

SEQ ID NO: 6 - protein, 14 aa, Artificial Sequence
  Synthetic peptide
GYCSRSIPPVCIPD

SEQ ID NO: 7 - protein, 14 aa, Artificial Sequence
  Synthetic peptide
GWCSRSYPPVCIPD

SEQ ID NO: 8 - DNA, 36 bp, Artificial Sequence
  Primer
cgcaattaaccctcagctagcggaacaaaagctggg

SEQ ID NO: 9 - DNA, 36 bp, Artificial Sequence
  Primer
ccgcctttgagtgagatgcatccgctcgccgcagcc

SEQ ID NO: 10 - DNA, 31 bp, Artificial Sequence
  Primer
gtcagtatgcatccgacaccatcgaatggtg

SEQ ID NO: 11 - DNA, 34 bp, Artificial Sequence
  Primer
gtcagtgctagcgccgaggcggaaaacatcatcg

SEQ ID NO: 12 - protein, 8 aa, Artificial Sequence
  Flag-tag
DYKDDDDK

SEQ ID NO: 13 - DNA, 34 bp, Artificial Sequence
  Primer
gtcagtgctagcatcggattataaagacgatgac

SEQ ID NO: 14 - DNA, 34 bp, Artificial Sequence
  Primer
gtcagttctagattattagcttgctttcgaggtg

SEQ ID NO: 15 - protein, 8 aa, Artificial Sequence
  Linker peptide
GGSGGSGG

SEQ ID NO: 16 - DNA, 55 bp, Artificial Sequence
  Adapter
ctagctggcgggtcgggtggatccggtggcgattataaagacgatgatgacaaac

SEQ ID NO: 17 - DNA, 55 bp, Artificial Sequence
  Adapter
tcgagtttgtcatcatcgtctttataatcgccaccggatccacccgacccgccag

SEQ ID NO: 18 - DNA, 43 bp, Artificial Sequence
  Primer
gctagttattgctcagcggtggcttgctttcgaggtgaatttc

SEQ ID NO: 19 - DNA, 85 bp, Artificial Sequence
  Designed oligonucleotide library
ccgccgcctcggcgctagcaggtnnktgtnnkarannknnkcctccgnnktgtnnkccggatggcgggtcgggtggatccggtgg

SEQ ID NO: 20 - protein, 14 aa, Artificial Sequence
  Synthetic peptide
GXCSXSXPPXCXPD

SEQ ID NO: 21 - protein, 14 aa, Artificial Sequence
  Synthetic peptide
GXCXXXXPPXCXPD

(19) United States
(12) Patent Application Publication
(10) Pub. No.: US 2012/0214748 A1
(43) Pub. Date: Aug. 23, 2012

(54) NOVEL PEPTIDES, PROCESS FOR PREPARATION THEREOF, AND USE THEREOF

(76) Inventors: Peter Gal, Budapest (HU); Gabor Pal, Budapest (HU); Kocsis Andrea Parisne, Szentes (HU); Peter Zavodszky, Budapest (HU)

(21) Appl. No.: 13/322,368

(22) PCT Filed: May 25, 2010
(86) PCT No.: PCT/HU2010/000061

§ 371 Date: Feb. 9, 2012

(30) Foreign Application Priority Data
May 25, 2009 (HU) P0900319

(51) Int. Cl. A61K 38/12; C07K 7/64; C12N 9/50; A61K 38/10; C12Q 1/37; C07K 7/08

(52) U.S. Cl. 514/21.1; 435/23; 530/327; 435/219; 514/21.5; 530/321

(57) ABSTRACT

The invention relates to peptides of the general formula (I) GX₁CSX₂SX₃PPX₄CX₅PD (SEQ ID NO: 20), where X₁ is Y, M, W, I, V, or A; X₂ is R or K; X₃ is Y, F, I, M, L, E, D, or H; X₄ is V, I, or H; and X₅ is I, V, Y, F, or W; and to the pharmaceutically acceptable salts, esters or prodrugs of the peptides according to general formula (I). In addition, the invention relates to pharmaceutical preparations, kits containing the preparations, and to procedures using the peptides and preparations.

This application includes biological sequence information in a Sequence Listing presented in an ASCII text file named “KOV104-SEQ-REV.txt”, created on Apr. 12, 2012, and having a file size of 5,164 bytes, which is incorporated herein by reference.

### FIELD OF THE INVENTION

The invention relates to novel peptides, especially oligopeptides, and it also relates to a process for the production of such peptides and to the use of such peptides in the production of medicaments.

### BACKGROUND OF THE INVENTION

The complement system is one of the most important components of the innate immunity of human and animal organisms. The complement system, as the immune system in general, is able to recognise, label and remove intruding pathogens and altered host structures (e.g. apoptotic cells). The complement system, as a part of the innate immune system, forms one of the first defense lines of the organism against pathogenic microorganisms, but it also links to the adaptive (acquired) immune system at several points forming a bridge, as it were, between innate and adaptive immune mechanism (Walport 2001a; Walport 2001b; Morgan 2005). The complement system is a network consisting of about 30 protein components, which components can be found in the blood plasma in soluble form, and also in the form of receptors and modulators (e.g. inhibitors) attached to the surface of cells. The main components of the system are serine protease zymogens, which activate each other in a cascade-like manner in strictly determined order. Certain substrates of the activated proteases are proteins containing a thioester bond (components C4 and C3 in the complement system). When these substrates are cleaved by the activated proteases, the reactive thioester group becomes exposed on the surface of the molecule, and in this way it is able to attach the cleaved molecule to the surface of the attacked cell. As a result of this, such cells are labeled so that they can be recognised by the immune system.

The biological functions of the complement system are extremely diverse and complex, and up till now they have not been explored in every detail. One of the most important functions is direct cytotoxic activity, which is triggered by the membrane attack complex (MAC) formed from the terminal components of the complement system. The MAP perforates the membrane of cells recognised as foreign, which results in the lysis and thereby destruction of such cells.

Another important function of the complement system is opsonisation, when the active complement components (e.g. Clq, MBL, C4b, C3b) settling on the surface of the cells promote the phagocytosis by leukocytes (e.g. macrophages). These leukocytes engulf the cells to be destroyed.

Furthermore, the inflammation initiation role of the complement system is also of outstanding importance. The cleavage products released during complement activation initiate an inflammatory process through their chemotactic stimulating effects on leukocytes (Mollnes 2002).

The components of the complement system are present in blood plasma in an inactive (zymogenic) form until the activation of the complement cascade is triggered by an appropriate signal (e.g. intrusion of a foreign cell, pathogen). The normal activity of the complement system is important from the aspect of maintaining immune homeostasis. Both its abnormal underactivity and its uncontrolled hyperactivity may result in the development of severe diseases or in the aggravation of already existing diseases (Szebeni 2004).

The complement system can be activated via three different pathways: the classical pathway, the lectin pathway and the alternative pathway. In the first step of the classical pathway the C1 complex binds to the surface of the activator, that is the biological structure recognised as foreign. The C1 complex is a supramolecular complex consisting of a recognition protein molecule (Clq) and serine proteases (C1r, C1s) associated to it (Arlaud 2002). First of all the Clq molecule binds to immune complexes, apoptotic cells, C-reactive protein and to other activator structures. As a result of the Clq molecule binding to the activator, the serine protease zymogens present in the C1 complex become gradually activated. In the tetramer C1s-C1r-C1r-C1s first the C1r zymogens autoactivate, then the active C1r molecules cleave and activate the C1s molecules. The active C1s cleaves the C4 and C2 components of the complement system, which cleavage products are the precursors of the C3-convertase enzyme complex (C4bC2a). The C3-convertase splits C3 components and transforms into C5-convertase (C4bC2aC3b). The C5-convertase cleaves C5, after which the activation of the complement system culminates in the terminal phase characteristic of all three pathways (formation of the MAC).

The activation of a different pathway of the complement system, the lectin pathway, is very similar to that of the classical pathway (Fujita 2004). However, in this case several different types of recognition molecules are involved: MBL (“mannose-binding lectin”) and ficolins (H, L and M types). These molecules bind to the carbohydrate structures on the surface of microorganisms. The binding of the recognition molecule is followed by the autoactivation of MASP-2 (“MBL-associated serine protease”-2) zymogen. The activated MASP-2 cleaves the C4 and C2 components, which results in the formation of the C3-convertase enzyme complex already described in the course of the classical pathway, and from this point the process continues as described above.

The alternative pathway starts with the cleavage of the C3 component and its anchoring to the surface of the biological structure recognised as foreign (Harboe 2008). If the C3b component created during the cleavage is bound to the cell membrane of a microorganism, then at the same time it also binds the zymogenic form of a serine protease called factor B (C3bB), which is activated by factor D present in the blood in active form, by cleavage. The C3bBb complex created in this way is the C3-convertase of the alternative pathway, which, after being completed with a further C3b molecule, transforms into C5 convertase. The alternative pathway may also be triggered spontaneously, independently, by the slow hydrolysis of the C3 component (C3w), but if either the classical or the lectin pathway gets to the point of C3 cleavage, the alternative pathway significantly amplifies their effect.

Of the pathways above, we describe the lectin pathway in greater detail, which has been recently discovered and has been characterized the least, and which is the most important from the aspect of the present invention. Several different types of proteases and non-catalytic proteins bind to the recognition molecules present in several different forms (MBL of different degrees of polymerisation and ficolins). MASP-2 even in itself is able to initiate the complement cascade (Ambrus 2003; Gal 2005), but this latter enzyme is present in a smaller amount (0.5 μg/ml) than MASP-1. The physiological function of the MASP-1 protease present in a higher amount (7 μg/ml) has not been completely explored yet.

Although MASP-1 on its own is not able to initiate the complement cascade (it can only cleave C2 but not C4), its activity may supplement the activity of MASP-2 at several points, therefore active MASP-1 may be necessary for amplifying and consummating the effect of the lectin pathway. Several signs indicate that to a certain extent MASP-1 is a protease similar to thrombin, forming a bridge between the two major proteolytic cascade systems—the complement system and the blood coagulation system—in the blood (Hajela 2002; Krarup 2008).

The gene of both MASP-1 and MASP-2 has an alternative splicing product. The MAp19 (sMAP) protein is produced from the MASP-2 gene, containing the first two domains of MASP-2 (CUB1-EGF). The MASP-3 mRNA is transcribed from the MASP-1 gene. The first five domains of MASP-3 are the same as the domains of MASP-1, but they differ in their serine protease domain. MASP-3 has low proteolytic activity on synthetic substrates, and its natural substrate is not known. Unlike other early proteases, it does not form a complex with the C1-inhibitor molecule. Probably the presence of both MAp 19 and MASP-3 acts against the activation of the lectin pathway, as these proteolytically inactive proteins compete with the active MASP-2 and MASP-1 enzymes for the binding sites on the recognition molecules.

As it has been mentioned above, abnormal operation of the complement system in the human or animal organism may result in developing disease. The uncontrolled activation of the complement system may result in damaging self-tissues, and developing inflammatory or autoimmune conditions (Beinrohr 2008). One of these conditions is ischemia-reperfusion (hereinafter: IR) injury, which occurs, when the oxygen supply of a tissue is temporarily restricted or interrupted (ischemia) for any reason (e.g. vascular obstruction), and after the restoration of blood circulation (reperfusion) cellular destruction starts. During reperfusion the complement system recognises ischemic cells as altered self cells and starts an inflammatory reaction to remove them. Partly this phenomenon is responsible for tissue damage occurring after cardiac infarction and stroke, and it may also cause complications during coronary bypass surgery and organ transplantations (Markiewski 2007). The lectin pathway probably plays a role in the development of IR injury. For this reason the deliberate suppression of the lectin pathway may reduce the extent and the consequences of IR injury. The lectin pathway may also become activated in the case of rheumatoid arthritis (hereinafter: RA) as MBL binds to the antibody form IgG-GO having altered glycosylation accumlated in the joints during RA. The uncontrolled activity of the complement system also plays a role in the development and maintenance of different neurodegenerative diseases (e.g. Alzheimer's, Huntington's and Parkinson's diseases, Sclerosis Multiplex), and it is one of the main factors in the pathogenesis of age-related macular degeneration (AMD) as well (Bora 2008). The latter clinical picture is responsible for half of all cases of age-related loss of eyesight in developed industrial countries. The complement system can also be associated with one of the forms of autoimmune nephritis (glomerulonephritis) and with another autoimmune disease, namely SLE (systemic lupus erythematosus).

If the complement system is inhibited during the first steps, the efficient and selective inhibition of certain activation pathways becomes possible without triggering general immunosuppression. By inhibiting MASP-1 and MASP-2 enzymes the lectin pathway can be blocked selectively (e.g. in the case of the diseases mentioned above), and by this the classical pathway responsible for the elimination of immunocomplexes is left untouched, that is functioning.

The C1r, C1s, MASP-1, MASP-2 and MASP-3 enzymes form an enzyme family having the same domain structure (Gal 2007). The trypsin-like serine protease (SP) domain responsible for proteolytic activity is preceded by five non-catalytic domains. The three domains CUB1-EGF-CUB2 forming the N-terminal part of the molecules (CUB=C1r/C1s, sea urchin Uegf and Bone morphogenetic protein-1; EGF=Epidermal Growth Factor) are responsible for the dimerization of the molecules (both in the case of MASP-1 and MASP-2) and for interacting with the molecules, e.g. for binding to the recognition molecules.

The C-terminal CCP1-CCP2-SP fragment (CCP=Complement Control Protein) of the molecules is equivalent to the whole of the molecule in respect of its catalytic properties. One of the characteristic features of complement proteases is that they have very narrow substrate specificity, they are able to cleave the well-defined peptide bonds of only a few protein substrates. Both the CCP modules and the SP domain contribute to this finely tuned specificity.

The SP domain contains the active centre characteristic of serine proteases, the substrate binding pocket and the oxyanion hole. Eight surface loop regions, the conformation of which is quite different in the different proteases, play a decisive role in determining subsite specificity.

On the one part the CCP modules stabilise the structure of the catalytic region, and on the other part they contain binding sites for large protein substrates. Although the small-molecule compounds generally used for inhibiting trypsin-like serine proteases (e.g. benzamidine, NPGB, FUT-175) inhibit the activity of complement proteases too (Schwertz 2008), this inhibition is not selective enough, it also extends to the inactivation of other serine proteases in the blood plasma, e.g. blood coagulation enzymes, kallikreins.

The only known natural inhibitor of the complement system, C1 inhibitor protein circulating in blood and belonging to the serpin family is also characterised by relatively wide specificity.

According to the state of the art no compounds or natural inhibitor proteins are known, which could efficiently and selectively inhibit the lectin pathway.

### SUMMARY OF THE INVENTION

The inhibition of the complement system, including the lectin pathway, may be an efficient tool in fighting against human and animal diseases occurring as a result of the abnormal activity of the complement system. However, presently no compound is available, with the use of which the complement system, primarily the lectin pathway, could be inhibited at the desired extent in order to combat such diseases. As it has been explained in detail above, the lectin pathway can be inhibited selectively by inhibiting the MASP-1 and MASP-2 enzymes.

For this reason we set the aim to develop compounds, which are able to inhibit selectively the lectin pathway of the complement system by inhibiting the MASP-1 and/or MASP-2 enzymes.

Surprisingly we found that the following peptides according to general formula (I) are suitable for the above objectives:

(SEQ ID NO: 20)
GX₁CSX₂SX₃PPX₄CX₅PD (I)

where

### X₁ is Y, M, W, I, V, A, and

### X₂ is R, K, and

### X₃ is Y, F, I, M, L, E, D, H, and

### X₄ is V, I, H, and

### X₅ is I, V, Y, F, W.

In accordance with the above, the invention relates to peptides according to general formula (I), their salts, esters and pharmaceutically acceptable prodrugs.

Especially preferably, the invention relates to peptides with the following sequences:

### GYCSRSYPPVCIPD (SEQ ID NO: 2),

### GICSRSLPPICIPD (SEQ ID NO: 3),

### GVCSRSLPPICWPD (SEQ ID NO: 4),

### GMCSRSYPPVCIPD (SEQ ID NO: 5),

### GYCSRSIPPVCIPD (SEQ ID NO: 6),

### GWCSRSYPPVCIPD (SEQ ID NO: 7), and

the cyclic version of the peptide with the sequence

### GICSRSLPPICIPD (SEQ ID NO: 3),

and their salts or esters.

Most preferably the invention relates to peptides with the sequence GYCSRSYPPVCIPD (SEQ ID NO: 2) and GICSRSLPPICIPD (SEQ ID NO: 3), their salts and esters.

Furthermore the invention also relates to pharmaceutical preparations, which contain at least one peptide according to general formula (I), its salt, ester or prodrug and at least one further additive. This additive is preferably a matrix ensuring controlled active agent release.

The invention relates especially to pharmaceutical preparations, which contain at least one of the peptides with the following sequences:

### GYCSRSYPPVCIPD (SEQ ID NO: 2),

### GICSRSLPPICIPD (SEQ ID NO: 3),

### GVCSRSLPPICWPD (SEQ ID NO: 4),

### GMCSRSYPPVCIPD (SEQ ID NO: 5),

### GYCSRSIPPVCIPD (SEQ ID NO: 6),

### GWCSRSYPPVCIPD (SEQ ID NO: 7),

the cyclic version of the peptide with the sequence GICSRSLPPICIPD (SEQ ID NO: 3), and/or their pharmaceutically acceptable salts and esters. Especially preferably the pharmaceutical preparation according to the invention contains peptides with the sequence GYCSRSYPPVCIPD (SEQ ID NO: 2) and GICSRSLPPICIPD (SEQ ID NO: 3), and/or their pharmaceutically acceptable salts and/or esters.

The invention also relates to kits containing at least one peptide according to general formula (I), its salt or ester.

The invention also relates to the screening procedure of compounds potentially inhibiting MASP enzymes, in the course of which a labeled peptide according to the invention is added to a solution containing MASP, then the solution containing one or more compounds to be tested is added to it, and the amount of the released marked peptide is measured. In this respect the MASP enzyme is preferably MASP-1 or MASP-2 enzyme.

The invention also relates to the use of peptides according to general formula (I) and their pharmaceutically acceptable salt or ester in the production of a pharmaceutical preparation suitable for curing diseases that can be cured by inhibiting the complement system. In accordance with this diseases can be selected preferably from the following group: inflammatory and autoimmune diseases, especially preferably ischemia-reperfusion injury, rheumatoid arthritis, neurodegenerative diseases, age-related macular degeneration, glomerulonephritis, systemic lupus erythematosus, and complement activation-related pseudo-allergy.

The invention also relates to a procedure for isolating MASP enzymes, in the course of which a carrier with one or more immobilised peptide according to general formula (I) are contacted with a solution containing a MASP enzyme and the preparation is washed. In this respect the MASP enzyme is preferably MASP-1 or MASP-2 enzyme.

Some of the above peptides according to the invention inhibit both MASP-1 and MASP-2 enzymes, others only inhibit the MASP-2 enzyme and not the MASP-1 enzyme. However, these peptides according to the invention inhibit thrombin, closely related to MASP enzymes, only in a very high concentration, and in general they only slightly inhibit trypsin too.

### BRIEF DESCRIPTION OF THE DRAWINGS

In the drawings

FIG. 1 shows a schematic representation of the phage display method;

FIG. 2 shows the checking of the result of the digestion described in example 1.1.3.2, performed on agarose gel (line 1 refers to the digested pMal-p2X lacIq gene, and line 2 refers to the digested pBlueKS-NheI-Nsi vector);

FIG. 3 shows the result of the test, in the course of which the vector and insert used for the ligation and transformation described in example 1.1.4.3 were examined to check concentration;

FIG. 4 shows a picture of the gel prepared in connection with the ligation test described in example 2.2.2;

FIG. 5 shows the sequence logo diagrams of the sequences obtained, where

FIG. 5.a shows the sequence diagram relating to the sequences selected from and specific to MASP-2;

FIG. 5.b shows the sequence diagram relating to the sequences selected from MASP-2, but also recognising MASP-1; and

FIG. 5.c shows the sequence diagram relating to the sequences selected from MASP-1, but also recognising MASP-2.

FIG. 6 shows the dose-related test results of the effect of the peptides according to the invention on blood coagulation, where

FIG. 6.a illustrates the experiment for measuring thrombin time, in the course of which plasma coagulation (fibrin formation) is triggered by adding thrombin to the plasma;

FIG. 6.b illustrates the experiment for measuring prothrombin time, in the course of which plasma coagulation (fibrin formation) is triggered by adding tissue factor to the plasma; and

FIG. 6.c illustrates the experiment for measuring activated thromboplastin time, which imitates the so-called “contact activated” or “intrinsic” pathway of blood coagulation;

FIG. 7 shows the effect of the peptides according to the invention on the three complement activation pathways, where

FIG. 7.a shows the effect of the selective “S” peptide, while

FIG. 7.b shows the effect of the non-selective “NS” peptide.

### DETAILED DESCRIPTION OF THE INVENTION

The present invention relates to peptides and peptide derivatives selectively inhibiting MASP-1 and MASP-2 (or only MASP-2) enzymes.

The present invention also relates to amino acid sequences, which are sequentially analogous to the described sequences and the biological activity of which is also analogous when compared to the described sequences. A person skilled in the art finds it obvious that certain side change modifications or amino acid replacements can be performed without altering the biological function of the peptide in question. Such modifications may be based on the relative similarity of the amino acid side chains, for example on similarities in size, charge, hydrophobicity, hydrophilicity, etc. The aim of such changes may be to increase the stability of the peptide against enzymatic decomposition or to improve certain pharmacokinetic parameters.

The scope of protection of the present invention also includes peptides, in which elements ensuring detectability (e.g. fluorescent group, radioactive atom, etc.) are integrated.

Furthermore, the scope of protection of the present invention also includes peptides, which contain a few further amino acids at their N-terminal, C-terminal, or both ends, if these further amino acids do not have a significant influence on the biological activity of the original sequence. The aim of such further amino acids positioned at the ends may be to facilitate immobilisation, ensure the possibility of linking to other reagents, influence solubility, absorption and other characteristics.

We used the IUPAC recommendations to mark the amino acid side chains in the given sequences (Nomenclature of α-Amino Acids, Recommendations, 1974—Biochemistry, 14(2), 1975).

The present invention also relates to the pharmaceutically acceptable salts of the peptides according to general formula (I) according to the invention. By this we mean salts, which, during contact with human or animal tissues, do not result in an unnecessary degree of toxicity, irritation, allergic symptoms or similar phenomena. As non-restrictive examples of acid addition salts the following are mentioned: acetate, citrate, aspartate, benzoate, benzene sulphonate, butyrate, digluconate, hemisulphate, fumarate, hydrochloride, hydrobromide, hydroiodide, lactare, maleate, methane sulphonate, oxalate, propionate, succinate, tartrate, phosphate, glutamate. As non-restrictive examples of base addition salts, salts based on the following are mentioned: alkali metals and alkaline earth metals (lithium, potassium, sodium, calcium, magnesium, aluminium), quaternary ammonium salts, amine cations (methylamine, ethylamine, diethylamine, etc.).

In respect of the present invention prodrugs are compounds, which transform in vivo into a peptide according to the present invention. Transformation can take place for example in the blood during enzymatic hydrolysis.

The peptides according to the invention can be used in pharmaceutical preparations, where one or more additives are needed to reach the appropriate biological effect. Such preparations may be pharmaceutical preparations combined for example with matrixes ensuring controlled active agent release, widely known by a person skilled in the art. Generally matrixes ensuring controlled active agent release are polymers, which, when entering the appropriate tissue (e.g. blood plasma) decompose for example in the course of enzymatic or acid-base hydrolysis (e.g. polylactide, polyglycolide).

In the pharmaceutical preparations according to the invention other additives known in the state of the art can also be used, such as diluents, fillers, pH regulators, substances promoting dissolution, colour additives, antioxidants, preservatives, isotonic agents, etc. These additives are known in the state of the art.

Preferably, the pharmaceutical preparations according to the invention can be entered in the organism via parenteral (intravenous, intramuscular, subcutaneous, etc.) administration. Taking this into consideration, preferable pharmaceutical compositions may be aqueous or non-aqueous solutions, dispersions, suspensions, emulsions, or solid (e.g. powdered) preparations, which can be transformed into one of the above fluids directly before use. In such fluids suitable vehicles, carriers, diluents or solvents may be for example water, ethanol, different polyols (e.g. glycerine, propylene glycol, polyethylene glycols and similar substances), carboxymethyl cellulose, different (vegetable) oils, organic esters, and mixtures of all these substances.

The preferable formulations of the pharmaceutical preparations according to the invention include among others tablets, powders, granules, suppositories, injections, syrups, etc.

The administered dose depends on the type of the given disease, the patient's sex, age, weight, and on the severity of the disease. In the case of oral administration the preferable daily dose may vary for example between 0.01 mg and 1 g, in the case of parenteral administration (e.g. a preparation administered intravenously) the preferable daily dose may vary for example between 0.001 mg and 100 mg in respect of the active agent.

Furthermore, the pharmaceutical preparations can also be used in liposomes or microcapsules known in the state of the art. The peptides according to the invention can also be entered in the target organism by state-of-the-art means of gene therapy.

If in order to reach the desired medical effect, an active agent selectively inhibiting MASP-1 or MASP-2 is needed, then from the peptides according to general formula (I) according to the invention the selective inhibitory peptides should be preferably selected. For example the peptide according to the invention selectively inhibiting the MASP-2 enzyme may be the peptide with the sequence GYCSRSYPPVCIPD (SEQ ID NO: 2), while the peptide according to the invention selectively inhibiting the MASP-1 enzyme may be the peptide with the sequence GICSRSLPPICIPD (SEQ ID NO: 3). In order to reach certain therapeutic aims it may be preferable to use a peptide inhibiting both MASP-1 and MASP-2, such as the cyclic peptide according to the invention with the sequence GICSRSLPPICIPD (SEQ ID NO: 3).

The peptides according to the invention can be preferably used in different kits, which can be used for measuring or localising different MASP enzymes (either in a way specific to any MASP enzyme, or both to the MASP-1 and MASP-2 enzymes at the same time). Such use may extend to competitive and non-competitive tests, radioimmunoassay, bioluminescent and chemiluminescent tests, fluorometric tests, enzyme-linked assays (e.g. ELISA), immunocytochemical assays, etc.

In accordance with the invention, kits are especially preferable, which are suitable for the examination of the potential inhibitors of MASP enzymes, e.g. in competitive binding assays. With the help of such kits a potential inhibitor's ability of how much it can displace the peptide according to the invention from a MASP enzyme can be measured. In order to detect it, the peptide according to the invention needs to be labelled in some way (e.g. incorporating a fluorescent group or radioactive atom).

The kits according to the invention may also contain other solutions, tools and starting substances needed for preparing solutions and reagents, and instructions for use.

The compounds (peptides) according to the invention according to general formula (I) can also be used for screening compounds potentially inhibiting MASP enzymes. In the course of such a screening procedure a peptide according to general formula (I) is used in a labelled (fluorescent, radioactive, etc.) form in order to ensure detectability at a later point. The preparation containing such a peptide is added to the solution containing MASP enzyme, in the course of which the peptide binds to the MASP enzyme. Following the appropriate incubation period, a solution containing the compound/compounds to be tested is added to the preparation, which is followed by another incubation period. The compounds binding to the MASP enzyme (if the tested compound binds to the surface of the enzyme partly or completely at the same site as the peptide, or somewhere else, but its binding alters the conformation of the MASP enzyme in such a way that it loses its ability to bind the peptide) displace the labelled peptide from the MASP molecule to the extent of their inhibiting ability. The concentration of the displaced peptides can be determined using any method suitable for detecting the (fluorescent or radioactive) labelling used on the peptide molecules. The incubation periods, washing conditions, detection methods and other parameters can be optimised in a way known by the person skilled in the art. The screening procedure according to the invention can also be used in high-throughput screening (HTS) procedures.

The peptides according to the invention can be used first of all in the medical treatment of diseases, in the case of which the inhibition of the operation of the complement system has preferable effects. Consequently the present invention also relates to the use of peptides in the production of medicaments for the treatment of such diseases. As it has been explained above in detail, such diseases are first of all certain inflammatory and autoimmune diseases, especially the following diseases: ischemia-reperfusion injury, rheumatoid arthritis, neurodegenerative diseases (e.g. Alzheimer's, Huntington's and Parkinson's disease, Sclerosis Multiplex), age-related macular degeneration, glomerulonephritis, systemic lupus erythematosus.

The compounds according to the invention can also be used for isolating MASP proteins, by immobilising peptides and contacting the preparation made in this way with the solution presumably containing MASP enzyme. If this solution really contains MASP enzyme, it will be anchored via the immobilised peptide. This procedure can be suitable both for analytical and preparative purposes. If the geometry of the binding of the given peptide on the MASP enzyme is not known, during this procedure a peptide anchored from several directions or even several peptides should be used to ensure appropriate linking. The solution containing the MASP enzyme can be a pure protein solution, an extract purified to different extents, tissue preparation, etc.

### Phage Display

The peptides according to the invention were developed using the phage display method.

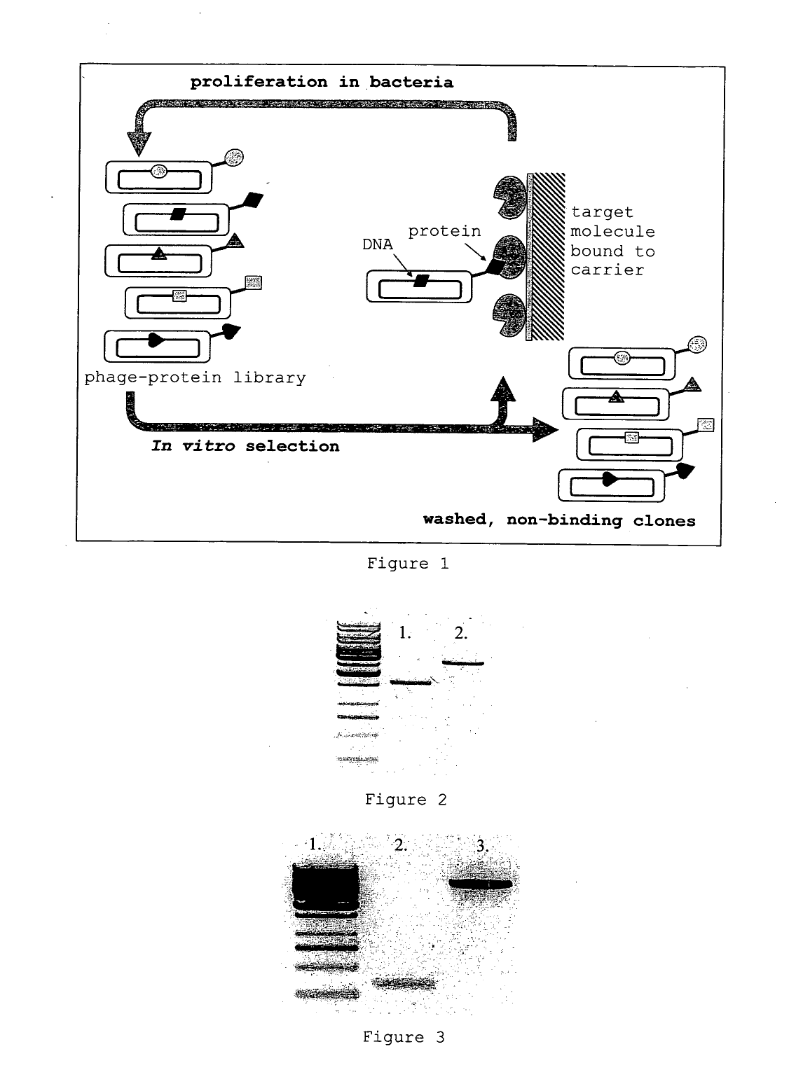

The phage display is suitable for the realisation of directed in vitro evolution, the main steps of the state-of-the-art procedure (Smith 1985) can be seen in FIG. 1. In the course of this the gene of the protein involved in evolution is linked to a bacteriophage envelope protein gene. In this way, when the bacteriophage is created, a fusion protein is produced, which becomes incorporated into the surface of the phage. The phage particle carries the gene of the foreign protein inside, while on its surface it displays the foreign protein. The protein and its gene are physically linked via the phage. For directed protein evolution, we change the codons of the gene coding it, carefully determined by us. Numerous codons can be changed at the same time using combinatorial mutagenesis based on a mixture of synthetic oligonucleotides. The position of the mutations and variability per position is determined at the same time.

After creating a DNA library containing several billions of variants and entering it into bacteria, the phage protein library is created. Each phage displays only one type of protein variant and carries only the gene of this variant. The individual variants can be separated from each other using affinity chromatography and analogue methods, on the basis of their ability to bind to a given target molecule chosen by the researcher (and generally linked to the surface). At the same time, as opposed to simple protein affinity chromatography, phage protein variants selected in this way have two important characteristic features. On the one part they are able to multiply, on the other part they carry the coding gene wrapped in the phage particle.

During the evolution, instead of examining individual mutants, in actual fact billions of experiments are performed simultaneously. Binding variants are multiplied, and after several cycles of selection-multiplication a population rich in functional variants is obtained. From this population individual clones are examined in functional tests, while the protein is still displayed on the phage. The phage protein variants found appropriate during the tests are identified by sequencing the physically linked gene. Besides the individual measurements, through the sequence analysis of an appropriately large number of function-selected clones it is also revealed what amino acid sequences enable fulfilling the function. In this way a database based on real experiments is prepared, which makes it possible to elaborate a sequence-function algorithm. The variants found the best on this basis are also produced as independent proteins, and these are examined in more accurate further tests.

### Creating a Library

The SFTI (Sun Flower Trypsin Inhibitor) molecule has a trypsin inhibitory activity and is a 14 amino acid peptide with the following sequence: GRCTKSIPPICFPD (SEQ ID NO: 1). In nature, that is in sunflower plants, it is created in a ring form, so the glycine marked as the N-terminal here and the asparagine acid marked as the C-terminal are linked by a peptide bond. The two cysteines form a disulphide bridge with each other. In vitro tests have demonstrated that if the disulphide bridge is intact, the above linear form is also a potent trypsin inhibitor (Korsinczky, 2001). Another special feature of the SFTI molecule is that structurally it is practically identical to the molecule part of significantly larger Bowman-Birk inhibitors interacting with enzymes (Luckett 1999; Korsinczky, 2001; Mulvenna 2005). The parts conserved in Bowman-Birk inhibitors and identical to the SFTI molecule are shown in boldface type: GRCTKSIPPICFPD (SEQ ID NO: 1). All boldfaced parts, except for one (Threonine in position 4), were kept while creating the library.

When designing the library the following randomisation scheme was used: GX¹CX²X³X⁴X⁵PPX⁶CX⁷PD (SEQ ID NO: 21). It is still the positions left unvaried for structural reasons that are shown in boldface type. In positions X¹, X², X⁴, X⁵, X⁶, and X⁷, all 20 natural amino acids were allowed, while in position X³ only the two basic amino acids (R/K) were allowed. The parts in italic were not varied, because on the basis of our first expectations we presumed that they do not get in contact with the protease.

In order to be able to select high-affinity binding molecules during phage display, it is essential that the binding molecule displayed should be presented in a low copy number per phage, ideally in one single copy (monovalent phage display). By this seemingly high-affinity binding (avidity) deriving from simultaneous binding to several anchored target molecules can be avoided. In the interest of this the SFTI library described above was expressed fused to a chymotrypsin inhibitor molecule, about which it had been demonstrated that when expressed linked to phage protein p8 it appears in one single copy per phage (Szenthe 2007). This is the Schistocerca Gregaria Chymotrypsin Inhibitor (SGCI) (Malik, 1999), about which we demonstrated in our preliminary experiments that it does not inhibit MASP enzymes, and it does not even bind to these enzymes.

Between a given element of the SFTI library and the SGCI molecule we also inserted a linear epitope tag recognisable by monoclonal antibodies, using an appropriate distance-keeping peptide link between the tag and the given element of the library. This was the so-called “Flag-tag”, which served two purposes. One of these was to be able to demonstrate easily the displaying of the library on the phage surface. The other purpose was to find out, after sequencing the clones obtained as a result of control selection using the antibody against the tag, clones of what sequence are obtained in the lack of the specific target enzymes, that is MASP1 and MASP2. In this way, when comparing the result of the selection performed on the enzymes to this group selected on the antibodies, the typical position-dependent amino acid preferences that can be really attributed to binding to the enzyme and are not the results of some other effect (e.g. more efficient production) can be revealed.

### EXAMPLES

Below the present invention is described in detail on the basis of examples, which, however, should not be regarded as examples to which the invention is restricted.

Through the examples a possible method of developing the phagemid system (example 1), preparing the library (example 2), phage selection (example 3) and the results (example 4) are shown. In example 5 peptide synthesis and the relating analytical tests are described.

### Example 1

### Developing the Phagemid System

### 1.1. Developing the Phagemid Vector

In the very first step, starting out from vectors available in commercial distribution, we developed our own phagemid vectors. For this we had to create new restriction endonuclease cleavage sites, which we realised using Kunkel mutagenesis (Kunkel, 1991).

### 1.1.1. Preparation of a Uracil-Containing Single-Stranded Kunkel-Template

### 1.1.1.1. Transformation

### Formulation:

0.5 μl pBluescript® II KS(−) phagemid (Stratagene, cat#212208-51.1 μg/μl, 2961 bp);

8 μl KCM solution [0.5 M KCl; 0.15 M CaCl₂; 0.25 M MgC1];
31.5 μl USP distilled water;
40 μl CJ236 K12 E. coli competent cell.

The transformant was incubated on ice for 20 minutes and then at room temperature for 10 minutes. We added LB medium of an amount ten times its volume (800 μl), and then it was shaken for 30 minutes at 37° C. at 200 rpm. Then a 100 μl amount was grown overnight at 37° C. on an LB-ampicillin plate [LB; 100 μg/ml ampicillin].

### 1.1.1.2. Infection

On the following day a colony was inoculated in 2 ml of medium [LB; 100 μg/ml ampicillin, 30 μg/ml chloramphenicol], and it was incubated overnight at 37° C., shaken at 200 rpm. Then 2 μl of the culture grown overnight was inoculated into 2 ml of medium of the same composition as above, and it was grown for 6 hours at 37° C., shaken at 200 rpm. Then it was infected with 30 μl M13KO7 helper phage (NEB, cat#N0315S), and then it was incubated at 37° C.-on, shaken at 200 rpm, for 40 minutes. The whole of the starter culture was transferred into 30 ml [2YT, 100 μg/ml ampicillin, 30 μg/ml chloramphenicol] medium. Phages were produced by growing the culture overnight at 37° C., shaken at 200 rpm, for 16-18 hours. On the following morning the culture was centrifuged at 8,000 for 10 minutes, at 4° C. The supernatant was transferred to clean tubes, and after adding a solution [2.5 M NaCl; 20% PEG-8000] of an amount of ⅕^{th} of its volume (6 ml) and incubating it for 20 minutes at room temperature, the phages were precipitated from the solution. The precipitate was centrifuged at 10,000 rpm for 20 minutes at 4° C., the supernatant was pipetted off. The precipitate was solubilized in 800 μl of PBS buffer.

The single-stranded plasmid was obtained from the phages using a Qiaprep® Spin™ M13 kit (Qiagen, cat#27704), according to the recipe attached to the kit, it was eluted from the column with 100 μl of ten times diluted EB buffer. The concentration of the product was checked in 35-times dilution at 260 nm (ssDNS OD260 nm=1=33 ng/μl). The concentration of the single-stranded uracil-containing pKS-phagemid vector obtained as a result of the above procedure was 407 μg/ml.

1.1.2. Introduction of Cleavage Sites Nsi and NheI using Kunkel Mutagenesis

### 1.1.2.1. Phosphorylation of Oligos

### Mutation Primers:

Blue-NheI-in-779 (36mer, SEQ ID NO: 8):
5′-cgcaattaaccctcagctagcggaacaaaagctggg-3′;
Blue-NsiI-in-1089 (36mer, SEQ ID NO: 9):
5′-ccgcctttgagtgagatgcatccgctcgccgcagcc-3′.

### Formulation:

2 μl 10× concentrated TM buffer [0.5 M Tris-HCl; 0.1 M MgCl₂; pH 7.5];
2 μl 10 mM ATP;
1 μl 100 mM DTT;
1 μl T4 polynucleotide kinase (Fermentas, 10u/μl);
36 ng Blue-NheI primer (4 μl)/36 ng Blue-Nsi primer (3.5 μl);
10 μl USP distilled water/10.5 μl USP distilled water.

The two phosphorylation reactions with the two primers separately were added together in a volume of 20 μl and incubated for 45 minutes at 37° C.

1.1.2.2. Hybridisation of oligonucleotides

The template: the proportion of primers was set so that the molar proportion is 1:3 in a volume of 25 μl.

### Formulation:

2.5 μl single-stranded Kunkel template (1 μg);
2 μl phosphorylated Blue-NheI-primer;
2 μl phosphorylated Blue-Nsi-primer;
2.5 μl 10× concentrated TM buffer;
16 μl USP distilled water.

The reaction mixture was heated for 1 minute in a 90° C. water bath, then it was immediately transferred into a 50° C. thermostat for another 3 minutes. Then it was centrifuged for a short time and placed in ice.

### 1.1.2.3. Preparation, Purification, Digestion of the Double-Stranded Product

After the hybridisation of oligonucleotides, with a second DNA synthesis a double-stranded product was in vitro produced, in which one of the strands contained uracil, it was the initial Kunkel template, but the other strand, which carries the mutation and was created by lengthening the primers, was free from uracil.

### Formulation:

1 μl 10 mM ATP;
1 μl 25 mM dNTP;
1.5 μl 100 mM DTT;
0.6 μl T4 ligase (NEB, 400 u/μl);
0.3 μl T7 polymerase (Fermentas, 10 u/μl).

The reaction mixture was incubated overnight at 14° C. The whole mixture was run on 1% agarose gel, isolated and purified with Qiaquick® Gel Extraction kit (Qiagen, cat#28704) according to the recipe. The product was eluted in 30 μl EB buffer and transformed into E. coli XL1 Blue competent cells according to the recipe mentioned above. These cells decompose the strand containing uracil, so in the bacteria grown in 3 ml cultures there are mainly clones, in which the vector was multiplied through the replication of the mutant strand not containing uracil. The double-stranded vector was isolated using Mini Plus™ Plasmid DNA Extraction system (Viogene, cat#GF2001) kit, in 50 μl EB buffer.

For the next step of genetic surgery the product was digested at the newly entered cleavage sites in 25 μl.

### Formulation:

20 μl vector miniprep;
2.5 μl 10× concentrated Y Tango™ buffer (Fermentas);
1.25 μl USP distilled water;
0.50 μl NheI (Fermentas, 10 u/ml);
0.75 μl Nsi (Promega, 10 u/ml).

Digestion took place at 37° C. overnight. The product was checked on 2% agarose gel using electrophoresis, and then, after isolating the digested plasmid from the gel using the method mentioned above, it was purified with the kit. The name of the vector obtained in this way is: pBlueKS-NheI-Nsi.

1.1.3 Adding the lacIq Gene

### 1.1.3.1 PCR

The lacIq gene and the maltose binding protein (MBP) signal sequence was isolated from the pMal-p2X vector (NEB, cat# N8077S, 200 μg/ml) using PCR.

### Primers:

pMal-lac-forward (SEQ ID NO: 10):
5′-gtcagtatgcatccgacaccatcgaatggtg-3′;
pMal-NheI-rev (SEQ ID NO: 11):
5′-gtcagtgctagcgccgaggcggaaaacatcatcg-3′.

### Formulation:

5 μl 10× concentrated Pfu buffer;
0.4 μl 25 mM dNTPs;
10 μl 25 mM MgSO₄;
0.5 μl pMal-p2X template;
0.5 μl 5 μM pMal-lac-forward primer;
0.5 μl 5 μM pMal-NheI-rev primer;
1 μl Pfu polymerase (Fermentas, 2.5 Wu/g1);
36.5 μl USP distilled water.

Program used during PCR:
1. 95° C. 180s;
2. 95° C. 45s;
3. 65° C. 45s;
4. 72° C. 240s;
5. 72° C. 480s.

Steps 2-4 were repeated twenty times.

### 1.1.3.2 Digestion

The product was purified using the GenElute™ PCR Clean Up kit (Sigma, cat#NA1020) according to the description, then it was digested overnight at 37° C. with restriction enzymes to make the sticky ends available needed for ligation.

### Formulation:

20 μl PCR product (lacIq gene);
2.5 μl 10× concentrated Y Tango™ buffer (Fermentas);
1 μl Nsi enzyme (=AvaIII, Fermentas, 10 u/μl);
0.5 μl NheI enzyme.

The digested PCR product was purified with a kit as above, and then together with the phagemid vector prepared, digested and purified in advance it was checked on 1% agarose gel. The results are show in FIG. 2, where line 1 corresponds to the digested pMal-p2X lacIq gene and line 2 corresponds to the digested pBlueKS-NheI-Nsi vector.

### 1.1.3.3. Ligation

### Formulation:

2 μl digested pBlueKS-NheI-Nsi vector;

6 μl digested pMal-p2X lacIq gene;

1 μl 10× concentrated T4 ligase buffer;

1 μl T4 ligase (Fermentas,1 Weiss u/μl).

Ligation was realised at room temperature, for 2 hours. Then the ligated product was transformed into 40 μl competent E. coli XL1 Blue cells as mentioned above. 100 μl of the transformed product [LB; 100 μg/ml ampicillin] was spread on an agar plate and incubated overnight at 37° C. From the developed colonies miniprep cultures were inoculated, and the plasmid was isolated using Viogene® kit. The ligation was checked with restriction digestion, for 1 hour at 37° C. For the EcoRI enzyme there is a cleavage site only inside the added lacIq gene.

### Formulation:

3.5 μl miniprep product;
1 μl 10×EcoRI buffer;
0.26 μl EcoRI enzyme (Fermentas, 10 u/μl);
5.24 μl USP distilled water.

On the basis of the 1% agarose gel it can be seen that digestion took place, that is ligation was successful. The name of the new phagemid vector is: pBlueKS-NheI-Nsi-lacIq.

### 1.1.4. Entering the Epitope Tag and the SGCI Part

### 1.1.4.1. PCR

The amino acid sequence of the Flag-tag used as an epitope tag is: DYKDDDDK (SEQ ID NO: 12). The SGCI part was fused to envelope protein p8, and the epitope tag was fused to the N-terminal of SGCI. As it has been mentioned above, the presence of SGCI ensures monovalent expression, so one phage will display a maximum of one library member peptide on its surface.

### Primers:

pGP8-Tag-NheI (SEQ ID NO: 13):
5′-gtcagtgctagcatcggattataaagacgatgac-3′;
P8-XbaI-rev (SEQ ID NO: 14):
5′-gtcagttctagattattagcttgctttcgaggtg-3′.

### Formulation:

5 μl 10× concentrated Pfu buffer;
8 μl 25 mM MgSO₄;
0.4 μl 25 mM dNTPs;
2 μl template: pGP8-Tag-SGCI vector (earlier construction);
0.5 μl 5 μM pGP8-Tag-NheI primer;
0.5 μl 5 μM P8-XbaI-rev primer;
1 μl Pfu polymerase (Fermentas, 2.5 u/μl);
36.2 μl USP distilled water.

Program used during PCR:
1. 95° C. 180s;
2. 95° C. 45s;
3. 60° C. 45s;
4. 72° C. 60s;
5. 72° C. 480s.

Steps 2-4 were repeated 25 times.

The PCR product was purified using a Sigma GenElute™ PCR Clean Up kit, according to the recipe.

### 1.1.4.2. Restriction Digestion

The pBlueKS-NheI-Nsi-lacIq vector was digested with restriction enzymes at 37° C. for 2 hours, to be able to ligate the Flagtag-SGCI part.

### Formulation:

2.5 μl pBlueKS-NheI-Nsi-lacIq miniprep;
3.5 μl 10× concentrated Tango™ buffer;
1.5 μl XbaI (Fermentas, 10 u/μl);
1.5 μl NheI (Fermentas, 10 u/μl);
3.5 μl USP distilled water.

The product was isolated from 1% agarose gel, purified with a Viogene® Gel-M™ kit and eluted in 45 μl of water. Then the product was treated with alkaline phosphatase at 37° C. for 45 minutes.

### Formulation:

43 μl digested pBlueKS-NheI-Nsi-lacIq vector, isolated from gel;
1 μl Shrimp Alkaline Phosphatase (SAP, Fermentas, 1 u/μl);
5 μl 10× concentrated SAP buffer.

The phosphatase was heat inactivated at 65° C. for 15 minutes.

### 1.1.4.3. Ligation and Transformation

Before preparing the reaction mixture, the vector and the insert was run on 1.8% agarose gel to check the concentration. The results are shown in FIG. 3.

In the figure the individual lines have the following meaning^{.}

1. 6 μl 1 kb DNA ladder (Fermentas);

2. Flagtag-SGCI-p8 PCR product; and

3. Digested, purified pBlueKS-NheI-Nsi-lacIq vector.

For the ligation the reaction mixture and the control products were incubated at room temperature for 90 minutes.

### Formulation:

2 μl pBlueKS-NheI-Nsi-lacIq vector;
7 μl Flagtag-SGCI-p8 PCR product;
1 μl 10× concentrated T4 ligase buffer;
1 μl T4 ligase (Fermentas, 1 Wu/μl).

The ligated product was transformed into competent E. coli XL1 Blue cells as mentioned above, spread and grown overnight at 37° C.

After inoculating 10 aliquots of media, 3 ml each, with individual bacterium colonies, a liquid culture grown overnight was prepared, and a double-stranded plasmid was isolated from them. The sticky ends generated by the XbaI and NheI enzymes are compatible with each other too, so from the ten clones the ones in the case of which integration was realised in the appropriate orientation were isolated by DNA sequencing, and the BigDye® Terminator v3.1 cycle Sequencing Kit (Applied Biosystems; cat#4336917) system was used for the PCR-reaction. The sequencing was run by BIOMI Kft. (Gödöllö). Among the 10 samples checked 2 good integrations were found. The name of the new vector is: pKS-Tag-SGCI-p8.

### 1.1.5. Integrating the Ser-Gly Adapter

For monovalent expression the following sequence of the functional units was created: library member-Ser/Gly/linker-Flagtag-SGCI-p8. For this, the pKS-Tag-SGCI-p8 vector was opened with NheI and XhoI enzymes, as a result of this step the original Flag-tag was omitted. Then the vector was ligated to an adapter containing a Gly-Ser linker (GGSGGSGG, SEQ ID NO: 15) and the Flag-tag, provided with the appropriate NheI and XhoI sticky ends. In order to check ligation a BamHI cleavage site was created inside the Flag-tag. This enzyme splits the appropriately ligated vector at two sites, the created product is 159 base pairs long, it could be detected using agarose gel electrophoresis.

### Formulation:

20 μl pKS-Tag-SGCI-p8 vector miniprep;
3 μl 10×Y Tango™ buffer;
2 μl XhoI (Fermentas, 10 u/μl);
5 μl USP distilled water.

The vector was digested at 37° C. for 2 hours, then on 0.8% agarose gel it was checked whether digestion was complete, as the given conditions were not ideal for the XhoI. Then 1 μl NheI enzyme was added to it and it was incubated at 37° C. for 1 hour. The product was isolated from agarose gel with a Viogene® Gel-M™ kit.

The adapters containing the linker and the Flag-tag were anellate to the digested vector.

### Adapters:

Ser-Gly-forward (SEQ ID NO: 16):
5′-ctagctggcgggtcgggtggatccggtggcgattataaagacgat
gatgacaaac-3′;
Ser-Gly-reverse (SEQ ID NO: 17):
5′-tcgagtttgtcatcatcgtctttataatcgccaccggatccaccc
gacccgccag-3′.

### Formulation:

15 μl digested pKS-Tag-SGCI-p8 vector;
2.8 μl 1.3 ng/μl Ser-Gly-forward primer;
1.7 ml 2.2 ng/μl Ser-Gly-reverse primer.

The reaction mixture was incubated at 90° C. for 1 minute and then at 50° C. for 3 minutes, centrifuged for a short time and placed on ice. For ligation the following was added to it:
2.2 μl 10× concentrated T4 ligase buffer;
1 μl T4 ligase (Fermentas, 1 Weiss u/μl).

Ligation was performed at 16° C. overnight. Competent E. coli XL1 Blue cells were transformed as described above, then the transformed product [LB; 100 mg/m1] was spread on plates. From the colonies starters were inoculated overnight, and with a Viogene® Mini-M™ kit miniprep plasmid was purified according to the instructions. The obtained samples were checked with DNA-sequencing, using the BigDye® Terminator v3.1 cycle Sequencing Kit, the PCR product was run by BIOMI Kft. (Gödöllö, Hungary).

In the following the library was created on the basis of the phagemid prepared in this way, its name is: pKS-SG-Tag-SGCI-p8.

### Example 2

### Preparing the Phage Library

The pKS-SG-Tag-SGCI-p8 vector checked with sequencing served as a template for creating the DNA library, which was created using polymerase chain reaction (PCR), with the help of a degenerated library oligo and a vector-specific oligo, as primers. The PCR product created in this way was integrated in the pKS-SG-Tag-SGCI-p8 vector.

### 2.1. PCR

### 2.1.1. Library Oligo

As it has been mentioned above, when planning the library the following randomisation scheme was used: GX¹CX²X³X⁴X⁵PPX⁶CX⁷PD (SEQ ID NO: 21). The SFTI-library was prepared so that 6 selected positions (X¹, X², X⁴, X⁵, X⁶, and X⁷) were completely randomised, that is the occurrence of all 20 amino acids was allowed, at position X³ only arginine and lysine was allowed (“R/K” position). Using the IUPAC codes relating to degenerated oligonucleotides, the oligonucleotide sequence of the library was the following (SEQ ID NO: 19):

5′-CC GCC GCC TCG GCG CTA GCA GGT [symbol] TGT [symbol] [symbol] [symbol] [symbol]
CCT CCG [symbol] TGT [symbol] CCG GAT
GGC GGG TCG GGT GGA TCC GGT GG-3′.

The part coding the peptide is shown in italics (i.e., nucleotides 21 through 62 of SEQ ID NO: 19), while randomised codons are marked in bold in SEQ ID NO: 19.

### 2.1.2 Preparing the DNA Library

The library was prepared using PCR, where one oligo carries the library member to be integrated, and the other oligo is a universal external primer. The entire reaction mixture, which amounted to 300 μl, was divided into 6 PCR tubes.

### Formulation:

30 μl 10× concentrated Taq buffer;
36 μl 25 mM MgCl₂;
2.4 μl 25 mM dNTP;
15 μl 13 μM SFTI-library oligonucleotide;
22 μl 10 μM pVIII 3′ primer;
9 μl (450 ng) pKS-SG-Tag-SGCI-p8 template;
180.6 μl USP distilled water;
5 μl Taq polymerase (Fermentas, 5 u/μl).
   The program:
1. 95° C. 60s;
2. 95° C. 30s;
3. 50° C. 30s;
4. 72° C. 60s;
5. 72° C. 120s.

Steps 2-4 were repeated 15 times.

The PCR product was checked on 1.5% agarose gel, then it was digested with Exol enzyme to remove the primers. It was incubated with 1 μl Exol enzyme per tube at 37° C. for 45 minutes, and then it was inactivated at 80° C. In order to multiply homoduplexes a short polymerisation cycle was inserted, the primer is a generally used external primer.

pVIII 3′ (SEQ ID NO: 18):
5′-gctagttattgctcagcggtggcttgctttcgaggtgaatttc-3′.

The following were added to each tube:
2.5 μl 2.5 mM dNTP;
1 μl 100 μM pVIII 3′ primer;
0.8 μl Taq polymerase (Fermentas, 5 u/μl).

The program is the same as in the case of the previous PCR, but only 2 cycles were run. The product was checked again on 1.5% agarose gel, then it was digested with Exol enzyme, and the content of the 6 PCR tubes was purified on 3 columns with a Sigma PCR Clean up kit according to the recipe. Elution took place in a volume of 52 μl/column, in EB buffer diluted 10×.

2.2 Integration of the DNA Library in the pKS-SG-Tag-SGCI-p8 Phagemid Vector

### 2.2.1 Digestion

The vector and the DNA library serving as an insert were digested in two steps, first they were cleaved with NheI enzyme. The unnecessary part splitting off during the digestion of the DNA library could not be removed from the reaction mixture, because it was nearly completely of the same size as the product. In order to prevent this piece from getting into the vector, Sad enzyme was also added in the first step of the digestion. Near the end of the unnecessary part it splits off a small fragment, which can be removed by purification, and the larger piece remaining there cannot be ligated with the sticky end of the Sad. Incubation was performed at 37° C., for 8 hours, and overnight.

### Formulation:

93 μl pKS-SG-Tag-SGCI-p8 vector (40 μg);
15 μl 10×Y Tango™ buffer;
4 μl NheI enzyme (Fermentas, 10 u/μl);
38 μl USP distilled water;

### (V=150 μl);

35 μl DNS-library PCR product;
15 μl 10×Y Tango™ buffer;
4 μl NheI enzyme (Fermentas, 10 u/μl);
4 μl Sad enzyme (Fermentas, 10 u/μl);
38 μl USP distilled water;

### (V=150 μl).

In the following, twice the amount of the Acc651 (=KpnI) enzyme producing the other sticky end was added. The concentration of the Tango™ buffer was also doubled.

To the digested pKS-SG-Tag-SGCI-p8 vector:
8 μl Acc651 (Fermentas, 10 u/μl);
19.8 μl 10× concentrated Tango™ buffer.

To the digested DNA-library:
8 μl Acc651 (Fermentas, 10 u/μl);
11 μl 10× concentrated Y Tango™ buffer.

### 2.2.2 Ligation

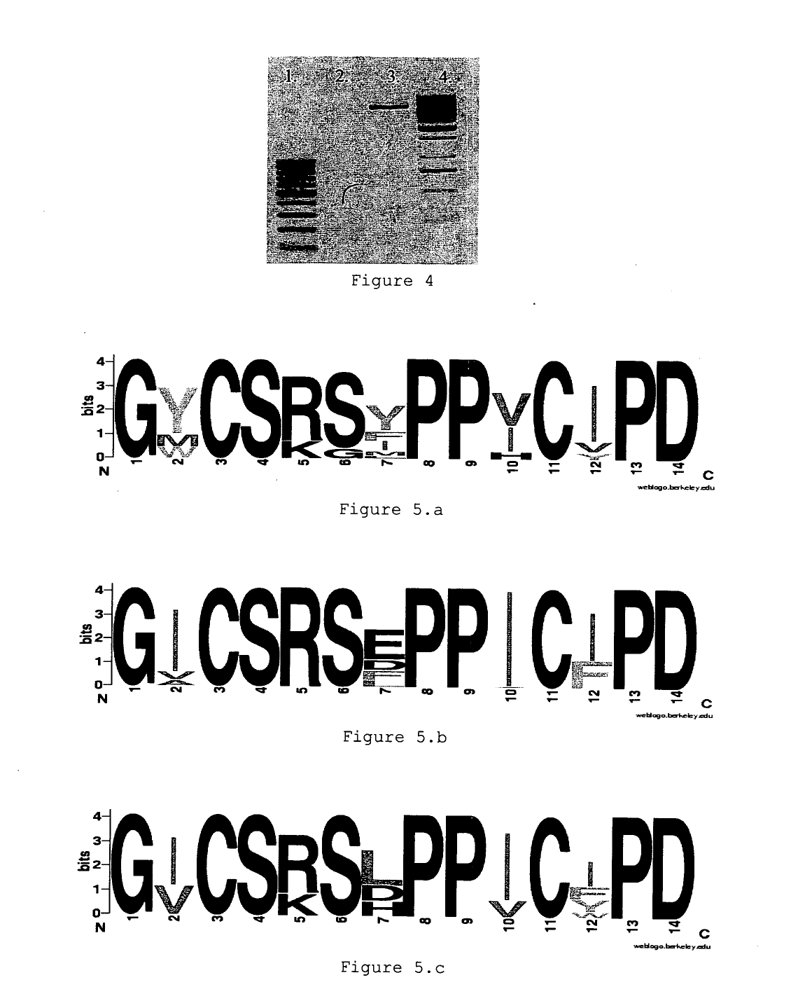

First both digested products were isolated from gel. The vector was isolated from 0.8% agarose gel, divided into six pockets, and then purified on 6 columns using a Viogene® Gel-M™ kit. The DNA-library was isolated from 1.8% gel and purified on 3 columns (FIG. 4). The lines of the gel image shown in the figure have the following meaning:
1. 1 μl 100 by DNA ladder;
2. 1 μl purified DNA-library;
3. 1 μl purified vector;
4. 5 μl 1 kb DNA ladder.

All samples were used for ligation, they were divided into 6 tubes and incubated for 18 hours at 16° C.:
210 ml purified pKS-SG-Tag-SGCI-p8 vector;
100 ml purified SFTI DNA-library;
2 ml T4 ligase (NEB, 400,000 ul/ml);
35 ml USP distilled water.

The product was purified with a Qiagen® Gel Elute™ kit, it was not isolated from gel only purified on the column. Elution was performed in 2×60 μl USP distilled water.

### 2.3 Electroporation, Multiplication of the Phage Library

The library was introduced to the supercompetent cells via electroporation. Our aim was to introduce the plasmid to as many cells as possible, so that our library contains 10⁸-10⁹ pieces. The DNA library, which is situated in USP distilled water so it is salt-free, was added to 2×350 ml supercompetent cells. The operation was performed in a cuvette with a diameter of 0.2 cm, according to the following protocol: 2.5 kV, 200 ohm, 25 μF.

After electroporation the cells were carefully transferred into 2×25 ml of SOC medium, incubated for 30 minutes at 100 rpm, at 37° C., then a sample was taken, a sequence was diluted from it and dripped onto [LB], [LB; 100 μg/ml ampicillin] and [LB; 10 μg/ml tetracycline] plates, and it was grown overnight at 37° C. The same procedure was followed in the case of non-electroporated control products and control products electroporated with water. After taking a sample, the 2×25 ml culture was infected with 2×250 μM13KO7 helper phage, shaken at 37° C. for 30 minutes at 220 rpm, and then the whole product was inoculated. The 2×250 ml [2YT; 100 μg/ml ampicillin; 30 μg/mlkanamycin] culture was grown in two 2-litre flasks at 37° C., at 220 rpm, for 18 hours.

On the basis of titration our library contained 1.2×10⁹ variants.

### Example 3

### Phage Selection

In the example below we demonstrate the selection of the library constructed according to the above examples, on MASP-1 and MASP-2 target enzymes.

### 3.1 The Target Enzymes

Human MASP-targets consist of a serine-protease (SP) domain and two complement control protein domains (CCP-1,-2) (Gal 2007). These are recombinant fragment products, which carry the catalytic activity of the entire molecule. The proteins were produced in the form of inclusion bodies, from which the conformation with biological activity was obtained by renaturation. Purification was performed by anion and cation exchange separation. The activity of the proteins was tested in a solution and also in a form linked to the ELISA plate. Production is described in detail in a different study (Ambrus 2003). The data of the targets used during selection:

MASP-1 CCP1-CCP2-SP: Mw=45478 Da, c_{stock}=0.58 g/l (hereinafter MASP-1);
MASP-2 CCP1-CCP2-SP: Mw=44017 Da, c_{stock}=0.45 g/l (hereinafter MASP-2);
Anti-Flagtag antibody: c_{stock}=4 g/l, (Sigma, Monoclonal ANTI-FLAG M2 antibody produced in mouse, cat# F3165).

### 3.2 Steps of Selection

### 3.2.1 Isolating the Phages

At the end of the operation described in chapter 2.3, phages were produced in 2×250 ml of culture for 18 hours. In the first step of the selection they were isolated to be able to use the library immediately for display.

The cell culture was centrifuged at 8,000 rpm for 10 minutes, at 4° C. The supernatant, which contained bacteriophages, was poured into clean centrifuge tubes, and a precipitating agent ⅕^{th} of its volume was added to it [2.5 M NaCl; 20% PEG-8000]. Precipitation took place at room temperature, for 20 minutes. Then it was centrifuged again at 10,000 rpm for 15 minutes, at 4° C. The supernatant was discarded, it was centrifuged again for a short time, and the remaining liquid was pipetted off. The white phage precipitate was solubilized in 25 ml [PBS; 5 mg/ml BSA; 0.05% Tween 20® surfactant] buffer. In order to remove possible cell fragments it was centrifuged again, the supernatant was transferred into clean tubes.

### 3.2.2. The First Selection Cycle

a) Immobilisation: The target molecules were immobilised on a 96-well Nunc Maxisorp™ ELISA plate (cat#442404). During immobilisation the concentration of MASP-1 and -2 was 20 μg/ml, and the concentration of the anti-Flag-tag antibody was 2 μg/ml. Proteins were diluted in the immobilisation buffer [200 mM Na₂CO₃; pH 9.4], and 100 μl was put in the wells. The period of immobilisation was optimised per protein. MASP-1 was incubated while mixing at 110 rev/min. at room temperature for 60 minutes, the antibody was incubated for 30 minutes, and MASP-2 was incubated overnight at 4° C. In the first selection cycle 12 wells per target protein were used. Every second row was left empty. As negative control only immobilising buffer was put in one row. This row was then treated the same way as the ones covered with target protein.
b) Blocking: The immobilising solution was removed, and 200 μl/well of blocking buffer [PBS; 5 mg/ml BSA] was put onto the plate. It was incubated at room temperature, for at least 1 hour, while mixing it at 150 rev/min.
c) Washing: The ELISA-plate was washed 4 times using 1 l of wash buffer [PBS; 0.05% Tween 20® surfactant].
d) Selection: The phages of the library isolated as described above were pipetted onto the plate, 100 μl in each well. It was incubated at room temperature, while mixing it at 110 rev/min., for 2.5 hours.
e) E. coli XL1 Blue culture: During the term of the selection, XLI Blue cells were inoculated from a plate freshly picked in advance using an inoculating loop, into 2×30 ml [2YT; 10 μg/ml tetracycline] of medium. These cells will be infected at a later point with phages eluted from the target proteins. At the time of infection the cells must be in the phase of exponential growth. A culture with OD₆₀₀ₙₘ˜0.3-0.5 was needed, which was obtained by growing it at 37° C., at 220 rpm, for 2-3 hours.
f) Washing: The ELISA-plate was washed 12 times using 3 litres of wash buffer.
g) Elution: Elution was performed using 100 mM HCl solution, 100 μl/well. The acid was applied, shaken for 5 minutes, and then it was drawn from each well one by one. The phages eluted from the individual target proteins were collected in a tube, in which 12×15 μl 1 M Tris-base buffer had been put in advance to quickly neutralise the acid solution containing the phages. The tubes were immediately mixed and placed on ice.
h) Infection: 4.5 ml of XL1 Blue culture in the phase of exponential growth was put in test tubes, and it was infected with 500 μl of phage solution eluted from the target protein. A total number of 4 infections was performed, with phages eluted from MASP-1 and MASP-2, from the antibody and from the negative control substance. The cultures were incubated at 37° C., at 220 rpm, for 30 minutes.
i) Titration: A 20-μl sample was taken from each infected culture, it was diluted to 10 times its volume with 2YT medium, and a sequence was prepared with further 10×dilutions. From each point 10 μl [LB; 100 μg/ml ampicillin] was dripped onto a plate and grown overnight at 37° C.
j) Infection with helper phage: Directly after sampling, 50 μM13KO7 helper phage was added to each culture in the test tubes, and they were incubated for a further 30 minutes.
k) All infected cultures were transferred into 3×200 ml [2YT; 100 μg/ml ampicillin; 30 μg/ml kanamycin] medium and incubated at 37° C., while mixing it at 220 rpm, for 18 hours. The control substance was not treated any further, it was only needed for titration.
l) Enrichment: On the following morning titration was checked, and after only one selection cycle a large difference could be detected as compared to the control substance. The number of phages eluted from the antibody was higher by 4 orders of magnitude than the number of phages eluted from the background, in the case of MASP the difference was 1-1.5 orders of magnitude.

### 3.2.3. The Second Selection Cycle

In this cycle the same steps were repeated as in the case of the first selection cycle, but in the blocking and wash buffer 2 mg/ml casein (Pierce, cat#37528) was used instead of BSA. By this modification the multiplication of phages binding to BSA can be avoided. In this step each target protein has its own control substance (12 wells), and the phages eluted and multiplied in the previous cycle were placed on each target protein.

The phages produced for 18 hours were isolated as described above, but at the end they were solubilized in 10 ml of sterile PBS buffer. The concentration of the phage solutions was measured at 268 nm, and then they were diluted with [PBS; 2 mg/ml casein; 0.05% Tween 20®] buffer so that each of them has a uniform OD₂₆₈ value of 0.5, and this is how they were used in the step of introduction. After the second selection cycle 2.7 ml of fresh exponentially growing XL1 Blue cells was infected with 300 μl of eluted phage. Titration was performed in all six cases (3 target proteins+3 control substances), and then the cultures also infected with helper phage were transferred into 30 ml [2YT; 100 μg/ml ampicillin; 30 μg/mlkanamycin] medium.

After the second selection cycle we obtained an enrichment of 10⁴ times in respect of the anti-flagtag antibody, 10 times in respect of MASP-1, 20 times in respect of MASP-2.

### 3.2.4 The Third Selection Cycle

Everything was performed in the same way as in the case of the second cycle, casein was also kept in the buffers. After isolation the phages were solubilized in 2.8 ml of sterile PBS, and for display they were diluted to 0D₂₆₈˜0.5.

After the third selection cycle enormous enrichment values were obtained as compared to the control substances. The difference was 10⁵ timeson the anti-flagtag antibody, and 10⁴ times on both MASP-s.

### 3.3. Testing Individual Clones Using Phage ELISA Assay

In this test we examined in what proportion of selected individual clones are able to bind to the target protein, while they do not display signals on the background.
a) Infection: In the case of MASP-1 and MASP-2 10 μl of eluted phage from selection cycle 2 and 3 was added to 90 μl of XL1 Blue culture in exponential phase. It was incubated for 30 minutes at 37° C. while mixing it at 220 rpm, then a 20-μl amount was taken out and 180 μl of 2YT medium was added to it. This dilution by 10 times was repeated two more times. From each dilution sequence we spread 100 μl on [LB; 100 μg/ml ampicillin] plates, and they were grown overnight at 37° C. The phages eluted from the anti-flagtag antibody in the first selection cycle were diluted first, and only after this were the cells infected. The reason for this was that the antibody can be much more preferably immobilised on the surface of the ELISA plate, and so much more phages were eluted. Due to the high phage concentration there is the risk of one cell being infected by several phages, which results in a mixed, incomprehensible sequences.
b) Injection: into so-called “single loose” tubes, into 500 μl of medium [2YT; 100 μg/ml ampicillin; 50 μM13KO7 helper phage] individual colonies were inoculated. These tubes are arranged similarly to a 96-well ELISA-plate arrangement, they move individually, so in a plate incubator, at 37° C., while mixing at 300 rev/min they are suitable for producing small-volume cultures.
c) Immobilisation: MASP-1 and MASP-2 proteins were immobilised in a concentration of 0.01 μg/μl, while the anti-flagtag antibody in a concentration of 1 μg/ml, in a volume of 100 μl/well, as described above in connection with selection, on Nunc ELISA Maxisorp® plates. Each clone was tested on its own target protein, on the background and on anti-Flag-tag antibody.
d) After 18 hours the tubes were centrifuged in a plate centrifuge at 2,500 rpm, for 10 minutes, at 4° C., the supernatant was pipetted into clean tubes. After ELISA the remaining supernatant was heated for 2 hours at 65° C., and after this they can be stored at −20° C., and they can be used for sequencing.
e) Blocking: The liquid was removed from the immobilised samples, and 200 μl/well of [PBS; 2 mg/ml casein] blocking buffer was placed in each well. Incubation took place at room temperature, for at least 1 hour, while mixing at 150 rev/min.
f) Washing: The plate was washed 4 times using 1 litre of wash buffer.
g) Phage application: The phages produced and isolated as described above were diluted by 2 times using [PBS; 2 mg/ml casein; 0.05% Tween 20® surfactant] buffer, and 100 μl was placed in the wells. From the same clone samples were pipetted into a total of 3 wells. Incubation was performed at room temperature, for 1 hour, while mixing at 110 rev/min.
h) Washing: The plate was washed 6 times using 1.5 litres of wash buffer.
i) Anti-M13 antibody: 100 μl of monoclonal anti-M13 HRP conjugated antibody (Amersham, cat#27-9421-01) diluted in [PBS; 2 mg/ml casein; 0,05% Tween 20® surfactant] buffer 10,000 times was placed in the wells, and then it was incubated for 30 minutes at room temperature, while mixing it at 110 rev/min.
j) Washing: The plate was washed 6 times with 1.5 litres of wash buffer, and then twice with PBS.
k) Development: 100 μl of 1-Step Ultra TMB-ELISA substrate (Pierce, cat#34028) diluted to twice its amount with USP distilled water was placed in each well, shaken for a while, and then the reaction was stopped by adding 50 μl of 1 M HCl in each well.
l) Reading: absorbance was measured at 450 nm, using BioTrak II (Amersham) plate reading photometer.

We took a sample from phage supernatants in the case of which the intensity of the background was low and which displayed at least three times more intensive signals on their own target protein, and prepared the samples for DNA sequencing. We used 2 μl of supernatant and used the BigDye® Terminator v3.1 cycle Sequencing Kit (Applied Biosystems; cat#4336917) system for the PCR reaction. It was run by BIOMI Kft. (Gödöllö). After interpreting the sequences it turned out that in the case of MASP-s further clones had to be selected and tested from the 2^{nd} selection cycle, as in the 3^{rd} cycle only a few individual sequences were found, only a few types were enriched. Our aim was to collect a multitude of sequences as diverse as possible to be able to construct a pattern about the amino acid preference of the target proteins.

### Example 4

### Results

In this example we describe the results of the tests described in examples 1-3, that is the sequences obtained.

From the phages eluted from MASP-1 we tested 32 clones using ELISA, and finally we found 9 individual sequences. In the case of MASP-2 we obtained 21 individual sequences from 80 ELISA points, while in the case of the anti-Flag-tag antibody we obtained 57 interpretable sequences from 72 tested clones.

When interpreting the results we had to take into consideration the effect of display-bias. A method for this is codon normalisation, as the NNK codon used for constructing the DNA library does not ensure the same frequency for the individual amino acids. The other, more realistic approach is normalisation with the data of the sequences selected from the antibody. Not all theoretically possible sequence types can be displayed on the surface of the phages, as some of them do not result in a realisable construction, or they represent too large a burden on the phage. However, from the antibody we obtained sequences that had occurred in reality, they were present at the initial step of the selection performed on the target proteins, so the forms specific to MASP-s were obtained from these.

After data normalisation we made sequence logo diagrams about the sequences with the help of WebLogo™ accessible on the internet (weblogo.berkeley.edu/logo.cgi; Crooks 2004 and Schneider 1990). We examined which were the preferred amino acids in the individual positions and how much they differed from each other depending on whether they derived from MASP-1 or MASP-2. We also compared our data with the sequence of the wild-type SFTI serving as a frame, which is the subnanomolar inhibitor of bovine trypsin.

The individual clones were examined in the ELISA system described above, on BSA used as background, on their own protein used as target, and also on the other MASP molecule to check possible cross reactions. On the basis of the results the sequences can be classified in three groups:

(a) sequences selected from and specific to MASP-2;
(b) sequences selected from MASP-2, but also recognising MASP-1; and
(c) sequences selected from MASP-1, but also recognising MASP-2.

We did not find any groups that recognised only MASP-1 specifically. The two non-selective groups (b and c) indicated very similar trends, no matter which MASP target they were selected on. On the horizontal axis of the sequence logo diagrams the number of the individual positions can be seen, site P1 corresponds to position 5. The sequence logo diagrams are shown in FIG. 5, where the number of the FIGS. 5.a; 5.b and 5.c) relate to the sequence logo diagrams of the groups marked (a), (b) and (c), in the same order. In each position the column height of the logo indicates how even the occurrence of the elements (20 different types of amino acids in our case) is. The less even this occurrence is, the higher the column. In the case of completely even distribution (all 20 amino acids occur in a proportion of 5%) the height is zero. The maximum value belongs to the case, when only one type of element (amino acid) occurs. Within the column the individual amino acids are arranged on the basis of the frequency of occurrence, the most frequent one is at the top. The height of the letter indicating the amino acid is in proportion with its relative frequency of occurrence in the given position (for example in the case of 50% frequency of occurrence, it is half the height of the column). In the case of colour diagrams, generally amino acids with similar chemical characteristics are shown in the same or in a similar colour, for which we used different shades of grey in the figure belonging to the present patent description.

With the help of the logo diagrams we determined the consensus sequence of the selective and non-selective groups, which we named M2-6E and M2-4G peptides on the basis of the name of the clone deriving from the selection, and their name reflecting their activity is “S” peptide (S for selective) or “NS” peptide (NS for non-selective) (see below).

MASP-2 selective M2-6E clone (SEQ ID NO: 2):
“S” peptide GYCSRSYPPVCIPD.
Non-selective M2-4G clone (SEQ ID NO: 3):
“NS” peptide GICSRSLPPICIPD.

The above peptides, and their point mutant and cyclic variants were produced via solid-phase peptide synthesis. The synthesis and the peptide analytical tests are described in example 5.

### Example 5

### Peptide Synthesis and Analysis

### 5.1. Peptide Preparation, Renaturation and Quality Inspection

Peptides were produced via solid-phase peptide synthesis using the standard Fmoc (N-(9-fluorenyl)methoxy carbonyl) procedure (Atherton 1989). Splitting off from the carrier and simultaneous removal of the protective group was performed using the TFA (trifluoroacetic acid) method, in the presence of 1,2-ethanedithiol, thioanisole, water and phenol, as radical-trapping agents. After the evaporation of the solution until nearly dry, the product was precipitated using cold diethyl ether. After dissolving the precipitate in water, volatile components were removed by lyophilisation. For renaturation, that is creating a disulphide bridge between the two cysteinyl side chains in the peptide, the lyophilised product was dissolved in water, in a concentration of 0.1 mg/ml. Oxidation was performed by mixing the solution besides continuous airing, the pH value was kept at an alkaline value (between 8-9) by adding N,N-diisopropyl-ethylamine. The complete realisation of oxidation was tested using reversed-phase HPLC and mass spectrometry. Isolation of the oxidised product in a more than 95% homogenous form was also performed using reversed-phase HPLC procedure. In the case of the M2-4G peptide the cyclic form was also produced, where peptide bond was created between the N and C terminal of the linear version. Cyclisation was performed as described below. Peptide synthesis took place on 2-ClTrt (2-chlorotrityl) resin, from where the peptide was split off using DCM (dichloromethane) solution containing 1% TFA. Under such conditions the side chain protective groups remain on the peptide. After the purification of the split off peptide using reversed-phase HPLC procedure, the linear peptide was dissolved using an amount of DMF (dimethylformamide), in the case of which the final concentration of the peptide was 0.1 mM. Then 1.1 equivalent of HATU (1-[Bis(dimethylamino)methylene]-1H-1,2,3-triazolo[4,5-b]pyridine-3-oxid hexafluorophosphate) and 3.0 equivalent of DIPEA (diisopropylethylamine) was added to it. After mixing the solution for 30 minutes at room temperature, the efficiency of cyclisation was tested using reversed-phase HPLC procedure and mass spectrometry. After the completion of cyclisation the sample was evaporated, and the peptide was purified using preparative reversed-phase HPLC procedure.

In the case of each isolated peptide quality control was performed by using the mass spectrometry procedure. Mass spectrometry analysis took place with the HP1100 type HPLC-ESI-MS system, with the flow-injection method, using 10 mM ammonium-formiate, pH 3.5 solution. The device was set to the parameters below. Both the drying gas and the atomizing gas was nitrogen, the flow rate of the drying gas was 10 l/minute, its temperature was 300° C. The pressure of the atomizing gas was 210 kPa, capillary voltage was 3500 V. The total ion current (TIC) chromatogram was made in positive ion setting, in the range between 300-2000 mass/charge. The mass data was evaluated with Agilent ChemStation® software. The name, sequence and mass data of the individual inhibitors produced are shown in Table 1 below.

TABLE 1
The theoretical and measured molecular weights of a few peptide inhibitors
according to the present invention, produced by chemical synthesis.

| | | SEQ ID | Theoretical | Measured |
|---|---|---|---|---|
| Inhibitor | Sequence | NO: | mass (Da) | mass (Da) |
| wild-type SFTI | GRCTKSIPPICFPD | 1 | 1531.8 | 1531.5 |
| M2-6E | GYCSRSYPPVCIPD | 2 | 1554.7 | 1554.5 |
| M2-4G | GICSRSLPPICIPD | 3 | 1468.7 | 1468.3 |
| M2-4G cyclic | [GICSRSLPPICIPD] | 3 | 1450.7 | 1450.5 |
| M1-3E-Y12W | GVCSRSLPPICWPD | 4 | 1527.7 | 1527.5 |
| M2-6E-Y2M | GMCSRSYPPVCIPD | 5 | 1522.7 | 1522.7 |
| M2-6E-Y7I | GYCSRSIPPVCIPD | 6 | 1504.7 | 1504.8 |
| M2-6E-Y2W | GWCSRSYPPVCIPD | 7 | 1577.8 | 1577.5 |

In the sequences shown in Table 1 the positions randomised during library constructions are underlined, and the positions, in which the amino acid is different from the one in wild-type SFTI are marked in bold.

5.2. Determining the Kᵢ Constant with Synthetic Peptide Substrates

The inhibiting ability of peptides was measured first on MASP enzymes and on trypsin. The inhibiting ability of only two peptides (see later) showing the most promising inhibition data on MASP enzymes was measured on thrombin too.

5.2.1. Measurements with MASP Enzymes

The synthetic substrate used in the measurements was Z-L-Lys-SBzl hydrochloride (Sigma, C3647), from which a 10 mM stock solution was prepared. The reactions were performed in a volume of 1 ml, at room temperature, in a buffer consisting of [20 mM HEPES; 145 mM NaCl; 5 mM CaCl₂; 0.05% Triton™-X100 surfactant]. The substrate cleaved by the enzyme entered into a reaction with the dithiodipyridine auxiliary substrate (Aldrithiol-4, Sigma, cat#143057) present in the solution in 2× excess. The release of the chromophore group created in this way was monitored in a spectrophotometer at 324 nm. A dilution sequence was prepared from the synthetic peptides, the enzyme was added to it, and it was incubated for 1 hour at room temperature. The concentration of the substrate and the length of the measuring period was chosen so that under the given conditions the enzyme should consume less than 10% of the substrate. In the course of measuring, a measuring method developed for the characterisation of tight-binding inhibitors was used (Empie, 1982). The incline of the straight line drawn on the initial phase of the reaction was normalised with the incline received in the case of the uninhibited enzyme reaction, and multiplied with the enzyme quantity. As a result of this we obtained the free enzyme concentration, which was shown as a function of the inhibitor concentration and drawn according to the following equation 1:

E=y=E₀−(E₀+x+Ki−(((E₀+x+Ki)̂2)−4*E₀*x)̂(1/2))/2, Equation 1,

where E is the free (uninhibited) enzyme concentration, and E₀ is the initial enzyme concentration. The MASP-1 MASP-2 concentration was determined by titration with C1 inhibitor. The results were calculated as the average of parallel measurements. The results are summarised in table 2 under point 5.3.

### 5.3. Measurements on Trypsin and Thrombin

The two consensus peptides, that is M2-6E and M2-4G proved to be the most promising MASP-2 and MASP-1 inhibitors, so we continued to characterise them by comparing them to the initial SFTI molecule in respect of their trypsin and thrombin inhibiting ability. In order to measure trypsin inhibition we used the measuring conditions described above, so the activity of trypsin was measured on Z-L-Lys-SBzl hydrochloride substrate as a function of the inhibitor peptide concentration. Evaluation took place as described above.

MASP enzymes perform their physiological task in the blood, so the possibility of using peptides depends on what effect they have on the activity of other proteases in the serum. We examined thrombin, the central enzyme of blood coagulation under similar conditions, but with Z-Gly-Pro-Arg-pNa substrate. The p-nitroanilide does not require an auxiliary substrate, the creation of the product can be monitored directly at 405 nm in a spectrophotometer. The measuring volume in a narrow cuvette was 350 μl, the concentration of the substrate was 505 μM. The thrombin was incubated for 20 minutes at room temperature with different inhibitor concentrations. The amount of thrombin was determined using the active-site titration method. Evaluation took place as described above. The results are summarised in Table 2 below.

TABLE 2
Summarising table of the enzyme inhibition of the individual
inhibitors. In the sequences shown the underlined and
bold letters have the same meaning as in Table 1.

| | K_{I}(nM) | | | | |
|---|---|---|---|---|---|
| Inhibitor | MASP-1 | MASP-2 | Thrombin | Trypsin | Seq., SEQ ID NO: |
| wild-type SFTI | NG | NG | 140000 | 0.1 | GRCTKSIPPICFPD, 1 |
| M2-6E | NG | 180 | 550000 | 1000 | GYCSRSYPPVCIPD, 2 |
| M2-4G | 65 | 1030 | 10000 | 260 | GICSRSLPPICIPD, 3 |
| M2-4G cyclic | 275 | 750 | — | 350 | [GICSRSLPPICIPD], 3 |
| M1-3E-Y12W | 140 | 5000 | — | 170 | GVCSRSLPPICWPD, 4 |
| M2-6E-Y2M | 4000 | 1500 | — | 4000 | GMCSRSYPPVCIPD, 5 |
| M2-6E-Y7I | NG | 7000 | — | 160 | GYCSRSIPPVCIPD, 6 |
| M2-6E-Y2W | NG | 580 | — | 1700 | GWCSRSYPPVCIPD, 7 |

Where it is not indicated otherwise, the inhibitors have an open chain. The sign “NG” means that the inhibition could not be measured even in the case of the highest inhibitor concentration used. Sign “-” means that no measurement was performed in respect of the given enzyme/inhibitor pair.

On the basis of the data it can be said that selective peptide (M2-6E, SEQ ID NO: 2) preferably inhibits MASP-2, it is not active on MASP-1, on trypsin its activity is lower by 4 orders of magnitude, and it is also a very poor thrombin inhibitor. As opposed to this, non-selective peptide (M2-4G, cyclic SEQ ID NO: 3) presents the features of a much more general inhibitor. It inhibits all four proteases, it is much weaker on trypsin than wild-type SFTI-1. It is a poor thrombin inhibitor, but as compared to the wild type its affinity has improved.

### 5.4. The Effect of Peptides on Blood Coagulation

We performed blood coagulation measurements using blood plasma taken from healthy individuals. From the blood obtained through venipuncture and treated with sodium citrate (3.8% wt/vol) the plasma was isolated by centrifugation (2000 g, 15 minutes, Jouan CR412 centrifuge).

Prothrombin time (PT) testing the extrinsic pathway of blood coagulation was measured on Sysmex® CA-500 (Sysmex, Japan) automatic system using Innovin® Reagent (Dale Behring, Marburg, Germany). Activated partial thromboplastin time (APTT) testing the intrinsic pathway of blood coagulation and thrombin time (TT) directly testing thrombin operation was measured on a Coag-A-Mate® MAX (BioMerieux, France) analyser using TriniClot™ reagent (Trinity Biotech, Wichlow, Ireland) and Reanal™ reagent (Reanal Finechemical, Hungary).

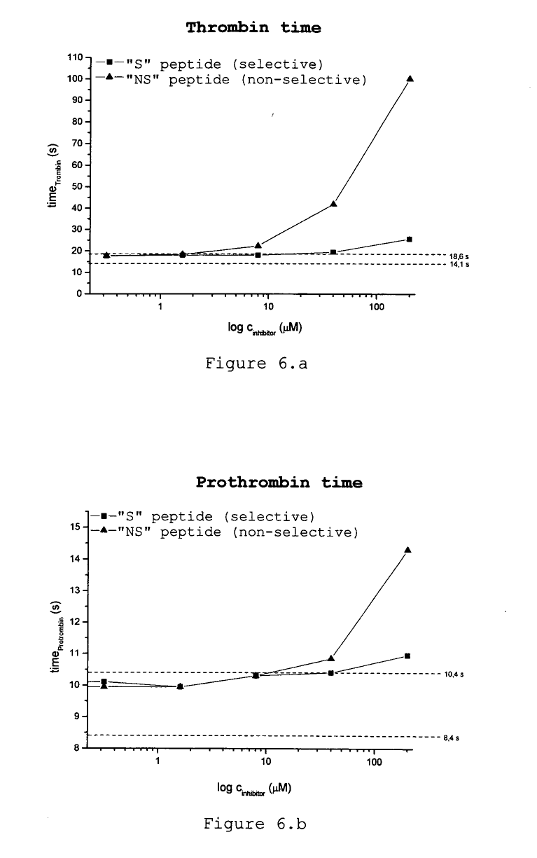

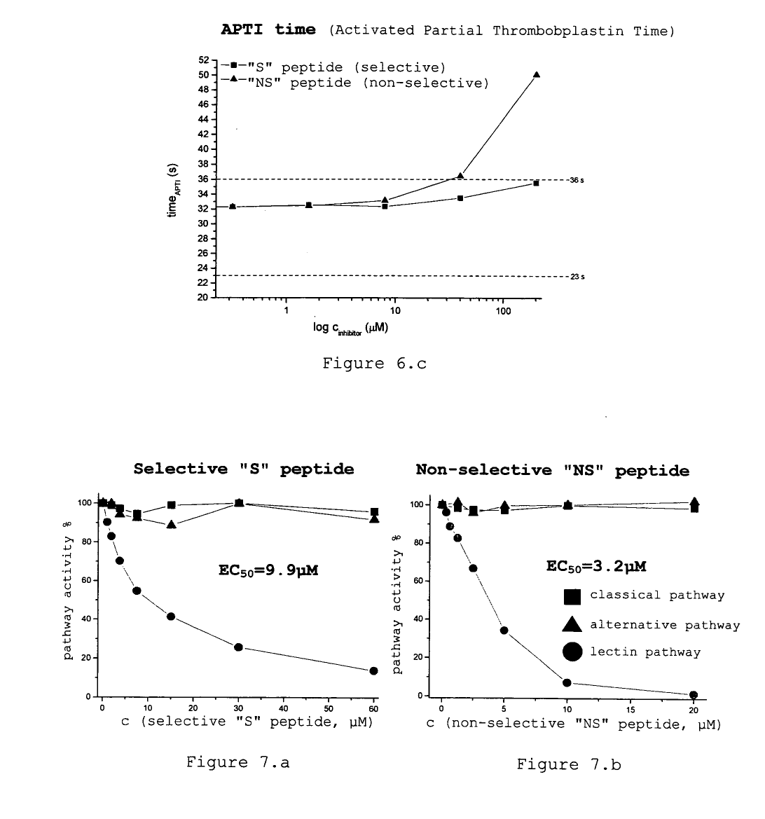

To examine the effects of peptides on blood coagulation we measured dose dependency, the results are shown on the graphs in FIG. 6. In each figure the area between the broken lines indicate the normal range relating to the given measurement. On the ordinates time is determined in seconds, while on the abscissas the logarithm of the inhibitor concentration is shown in μM.

FIG. 6.a illustrates an experiment for measuring thrombin time, in the course of which plasma coagulation (fibrin formation) is initiated by adding thrombin to the plasma. The effect of externally added thrombin is inhibited with peptide used in increasing concentrations (abscissa), and the time needed for coagulation is measured (ordinate). FIG. 6.b illustrates an experiment for measuring prothrombin time, in the course of which plasma coagulation (fibrin formation) is initiated by adding tissue factor to the plasma, as a result of which, through the activation of factor VII, the prothrombinase complex activating thrombin is created in several steps. In this experiment the external pathway of blood coagulation activated as a result of a trauma (vascular injury) is imitated. The members of the protease cascade initiated by the tissue factor are inhibited with peptide used in increasing concentrations (abscissa), and the time needed for coagulation is measured (ordinate). FIG. 6.c illustrates an experiment for measuring activated thromboplastin time, which imitates the so-called “contact activated or intrinsic” pathway of blood coagulation, which is initiated physiologically for example by the occurrence of collagen in the blood. In the experiment it is realised by adding a different large-surface material, for example kaolin powder, instead of collagen. As a result of this, through activating factor XII a protease cascade is initiated again, as a result of which the prothrombinase complex activating thrombin is created. The members of this protease cascade are inhibited with peptide used in increasing concentrations (abscissa), and the time needed for coagulation is measured (ordinate).

In the case of all three measuring occasions selective “S” peptide remained near the normal range even when the concentration was 200 μM, so from the aspect of MASP-inhibition it did not inhibit coagulation in relevant concentrations. As opposed to this non-selective “NS” peptide reached the extreme measuring value in the case of 200 μM, which means that it inhibited blood coagulation significantly. The data explained in the previous chapter have demonstrated that “NS” peptide inhibits thrombin at a Kᵢ value of 10 μM, which in itself explains its effect shown in the tests. In the last step of blood coagulation thrombin is the enzyme that splits fibrinogen, creating by this the fibrin-based coagulum. So the inhibition of thrombin in itself is enough for the efficient inhibition of blood coagulation. Because of this, on the basis of the blood coagulation tests above it cannot be decided whether the “NS” peptide relatively preferably inhibiting thrombin also inhibits the blood coagulation factors that precede thrombin from a functional aspect in the blood coagulation cascade (e.g. VIIA, IXa, Xa, XIa, XIIa). At the same time, the weaker effect of the selective “S” peptide on blood coagulation demonstrated in all three tests indicates that this peptide cannot be a potent inhibitor of the initial components of the cascade either.

### 5.5. The Effects of the Peptides According to the Invention on the Three Complement Activation Pathways

As it has been explained in detail above, the complement system can be activated through three pathways and it leads to the same single end-point. Three activation pathways include the classical, the lectin and the alternative pathway. MASP-s are the enzymes of the initial phase of the lectin pathway, so it is important to know what effect the MASP inhibitors according to the invention have on the lectin pathway, on the other two activation pathways and on the joint phase following the meeting of the three pathways.

For measuring we used the so-called WIELISA kit (Euro-Diagnostica AB, COMPL300) developed for the selective measuring of the complement pathways, on the basis of the instructions for use attached to the kit. The guiding principle of measuring is that according to the three activation pathways it uses three measuring conditions, in which the currently examined complement activation pathway can operate, while the other two pathways are inactive. At the same time, the product detected during measuring is not a pathway-selective component, but the last element of the joint section of the activation pathways, the C₅₋₉ complex.

For measuring, the blood sample was incubated for 1 hour at room temperature, then it was centrifuged and the serum was stored in small batches at −80° C. The serum was diluted according to the prescriptions with the buffer belonging to the given complement pathway, it was incubated for 20 minutes at room temperature, the dilution sequence prepared from peptides was added to it, it was incubated for 20 minutes at room temperature, then it was pipetted into the appropriate wells of a special ELISA plate. In the following, washing, incubation and antibody addition was performed according to the instructions for use. It was incubated for 20 minutes with the substrate, and then the data was read at 450 nm in a spectrophotometer. A parallel belonged to each measuring point, 100% activity was represented by the serum without an inhibitor. The measurements were performed at the same time and on the same plate, from one single melted serum sample.

The measurements lead to the extremely important result that “S” peptide and “NS” peptide are both efficient and specific inhibitors of the lectin pathway of the complement system. This result is in compliance with the result demonstrated earlier, according to which both peptides inhibit the MASP-2 enzyme very efficiently, which enzyme, according to our present knowledge, is responsible for the initiation of the lectin pathway.

Numerous serine proteases operate in the complement system, and some of them are very similar to the MASP enzymes. Despite this neither “S” peptide nor the “NS” peptide inhibited either the classical or the alternative pathway.

As in the course of measuring the classical and the alternative pathway the presence of the peptides according to the invention did not inhibit the creation of the terminal C₅₋₉ complex, it is for certain that the peptides according to the invention do not inhibit the proteases of the joint section of the complement system, so the inhibition of the lectin pathway really took place at the beginning of the lectin pathway, at the level of the MASP enzymes. It is worth pointing out that the IC50 data obtained in the course of the WIELISA measuring is about 30 times, 60 times higher than the Kᵢ values obtained in the course of MASP-2 inhibition measurements based on synthetic substrates. A possible explanation for this is the following: inhibitor peptides bind to the MASP-2 enzyme directly at the substrate binding site, and this binding successfully competes with the relatively weak interaction of small synthetic substrates with the same enzyme surface. However, besides the substrate binding site situated on the protease domain, physiological substrates can create bonds via other surfaces too (exosites), and they bind to the enzyme with a higher affinity than small synthetic substrates. It is because of this higher affinity that inhibitor peptides must be used in a higher concentration for the balance to be shifted from the enzyme-substrate complex towards the enzyme-inhibitor complex.

### LITERATURE REFERENCES

Ambrus, G., Gal, P., Kojima, M., Szilágyi, K., Balczer, J., Antal, J., Gráf, L., Laich, A., Moffatt, B., Schwaeble, W., Sim, R. B. and Zavodszky, P. (2003) Natural substrates and inhibitors of mannan-binding lectin-associated serine protease-1 and -2: a study on recombinant catalytic fragments. J. Immunol. 170, 1374-1382.
Arlaud, G. J., Gaboriaud, C., Thielens, N.M., Budayova-Spano, M., Rossi, V. and Fontecilla-Camps, J. C. (2002) Structural biology of the C1 complex of complement unveils the mechanisms of its activation and proteolytic activity. Mol. Immunol. 39, 383-394
Atherton, E.; Sheppard, R. C. (1989). Solid Phase peptide synthesis: a practical approach. Oxford, England: IRL Press. ISBN 0199630674.
Beinrohr, L., Dobó, J., Závodszky, P. and Gál, P. (2008) C1, MBL-MASPs and C1-inhibitor: novel approaches for targeting complement-mediated inflammation Trends in Molecular Medicine doi:10.1016/j.molmed.2008 Sep. 9
Bora, N. S., Jha, P. and Bora, P. S. (2008) The role of complement in ocular pathology. Semin. Immunopathol. 30, 85-95

Crooks G E, Hon G, Chandonia J M, Brenner S E. 2004. “WebLogo: A sequence logo generator”, Genome Research, 14:1188-1190
Dobó J., Harmat, V., Sebestyén, E., Beinrohr, L., Závodszky, P. & Gál, P. (2008). Purification, crystallization and preliminary X-ray analysis of human mannose-binding lectin-associated serine protease-1 (MASP-1) catalytic region. Acta Crystallogr Sect F Struct Biol Cryst Commun 64, 781-4.
Empie, M. W. & Laskowski, M., Jr. (1982). Thermodynamics and kinetics of single residue replacements in avian ovomucoid third domains: effect on inhibitor interactions with serine proteinases. Biochemistry 21, 2274-84.
Fujita, T., Matsushita, M. and Endo, Y. (2004) The lectin-complement pathway—its role in innate immunity and evolution. Immunol. Rev. 198, 185-202
Gal, P., Barna, L., Kocsis, A. and Zavodszky P. (2007) Serine proteases of the classical and lectin pathways: Similarities and differences Immunobiol. 212, 267-277
Gál, P., Harmat, V., Kocsis, A., Bián, T., Barna, L., Ambrus, G., Vegh, B., Balczer, J., Sim, R. B., Náray-Szabo, G., Závodszky, P. (2005) A true autoactivating enzyme. Structural insights into mannose-binding lectin-associated serine protease-2 activation. J. Biol. Chem. 280, 33435-33444.
Hajela, K., Kojima, M., Ambrus, G., Wong, K. H. N., Moffatt, B. E., Fergula, J., Hajela, S., Gal, P., Sim, R. B. (2002) The biological functions of MBL-associated serine proteases (MASPs). Immunbiol. 205, 467-475.
Harboe, M. and Mollnes, T. E. (2008) The alternative complement pathway revisited. J. Cell. Mol. Med. 12, 1074-1084
Harmat, V., Gál, P., Kardos, J., Szilagyi, K., Ambrus, G., Vegh, B., Náray-Szabó, G. & Závodszky, P. (2004). The structure of MBL-associated serine protease-2 reveals that identical substrate specificities of Cls and MASP-2 are realized through different sets of enzyme-substrate interactions. J Mol Biol 342, 1533-46.
Korsinczky, M. L., Schirra, H. J., Rosengren, K. J., West, J., Condie, B. A., Otvos, L., Anderson, M. A. & Craik, D. J. (2001). Solution structures by 1H NMR of the novel cyclic trypsin inhibitor SFTI-1 from sunflower seeds and an acyclic permutant. J Mol Biol 311, 579-91.
Krarup, A, Gulla, K. C., Gál, P. Hajela, K. and Sim R. B. (2008) The action of MBL associated serine protease 1 (MASP1) on factor XIII and fibrinogen. Biochim. Biophys. Acta. 1784, 1294-1300.
Kunkel, T. A., Bebenek, K. & Mcclary, J. (1991). Efficient Site-Directed Mutagenesis Using Uracil-Containing DNA. Methods in Enzymology 204, 125-139.
Laskowski, M., Jr. & Kato, I. (1980). Protein inhibitors of proteinases. Annu Rev Biochem 49, 593-626.
Luckett, S., Garcia, R. S., Barker, J. J., Konarev, A. V., Shewry, P. R., Clarke, A. R. & Brady, R. L. (1999). High-resolution structure of a potent, cyclic proteinase inhibitor from sunflower seeds. J Mol Biol 290, 525-33.
Malik, Z., Amir, S., Pál, G., Buzás, Z., Várallyay, E., Antal, J., Szilágyi, Z., Vékey, K., Asbóth, B., Patthy, A. & Gráf, L. (1999). Proteinase inhibitors from desert locust, Schistocerca gregaria: engineering of both P-1 and P-1′ residues converts a potent chymotrypsin inhibitor to a potent trypsin. Biochimica Et Biophysica Acta-Protein Structure and Molecular Enzymology 1434, 143-150.
Markiewski, M. M. and Lambris, J. D. (2007) The role of complement in inflammatory diseases—from behind the scenes into the spotlight. Am J. Pathol. 171, 715-727.
Mollnes, T. E., Song, W. C. and Lambris, J. D. (2002) Complement in inflammatory tissue damage and disease. Trends Immunol. 23, 61-64
Morgan, B. P., Marchbank, K. J., Longhi, M. P., Harris, C. L. and Gallimore, A. M. (2005) Complement: central to innate immunity and bridging to adaptive responses. Immunol. Lett. 97, 171-179
Mulvenna, J. P., Foley, F. M. & Craik, D. J. (2005). Discovery, structural determination, and putative processing of the precursor protein that produces the cyclic trypsin inhibitor sunflower trypsin inhibitor 1. J Biol Chem 280, 32245-53.
Schneider T D, Stephens R M. 1990. “Sequence Logos: A New Way to Display Consensus Sequences.” Nucleic Acids Res. 18:6097-6100
Schwertz H, Carter J M, Russ M, Schubert S, Schlitt A, Buerke U, Schmidt M, Hillen H, Werdan K, Buerke M. (2008) Serine protease inhibitor nafamostat given before reperfusion reduces inflammatory myocardial injury by complement and neutrophil inhibition. J Cardiovasc Pharmacol. 2008 August; 52(2):151-60.
Smith, G. P. (1985). Filamentous Fusion Phage—Novel Expression Vectors That Display Cloned Antigens on the Virion Surface. Science 228, 1315-1317.
Szebeni, J. ed. (2004) The complement system. Novel roles in health and disease Kluwer Academic Publishers, ISBN 1-4020-8055-7.
Szenthe, B., Patthy, A., Gáspári, Z., Kékesi, A. K., Gráf, L. & Pál, G. (2007). When the surface tells what lies beneath: Combinatorial phage-display mutagenesis reveals complex networks of surface-core interactions in the pacifastin protease inhibitor family. Journal of Molecular Biology 370, 63-79.
Walport, M. J. (2001a) Complement. First of two parts. N. Eng. J. Med. 344, 1058-1066.
Walport, M. J. (2001b) Complement. Second of two parts. N. Eng. J. Med. 344, 1140-1144.

1. Peptides A peptide according to general formula (I)
(SEQ ID NO: 20)
GX₁CSX₂SX₃PPX₄CX₅PD (I)
where
X₁ is Y, M, W, I, V, A, and
X₂ is R, K, and
X₃ is Y, F, I, M, L, E, D, H, and
X₄ is V, I, H, and
X₅ is I, V, Y, F, W;
and salts, esters and pharmaceutically acceptable prodrugs thereof.

2. The peptide according to claim 1, where the peptides are selected from peptides with the following sequences:
GYCSRSYPPVCIPD (SEQ ID NO: 2),
GICSRSLPPICIPD (SEQ ID NO: 3),
GVCSRSLPPICWPD (SEQ ID NO: 4),
GMCSRSYPPVCIPD (SEQ ID NO: 5),
GYCSRSIPPVCIPD (SEQ ID NO: 6),
GWCSRSYPPVCIPD (SEQ ID NO: 7), and
the cyclic peptide with the sequence
GICSRSLPPICIPD (SEQ ID NO: 3),
and their salts and esters.

3. The peptide according to claim 2, where the peptides are selected from peptides with the following sequences:
GYCSRSYPPVCIPD (SEQ ID NO: 2), and
GICSRSLPPICIPD (SEQ ID NO: 3),
and their salts and esters.

4. A pharmaceutical preparation, which contains at least one peptide according to general formula (I)
(SEQ ID NO: 20)
GX₁CSX₂SX₃PPX₄CX₅PD (I)
where
X₁ is Y, M, W, I, V, A, and
X₂ is R, K, and
X₃ is Y, F, I, M, L, E, D, H, and
X₄ is V, I, H, and
X₅ is I, V, Y, F, W;
and/or contains the pharmaceutically acceptable salt, ester or prodrug of a peptide according to general formula (I),
and at least one further additive.

5. The pharmaceutical preparation according to claim 4, characterised by that at least one of the additives is a matrix ensuring controlled active agent release.

6. The pharmaceutical preparation according to claim 4, characterised by that the peptide according to general formula (I) is selected from peptides with the following sequences:
GYCSRSYPPVCIPD (SEQ ID NO: 2),
GICSRSLPPICIPD (SEQ ID NO: 3),
GVCSRSLPPICWPD (SEQ ID NO: 4),
GMCSRSYPPVCIPD (SEQ ID NO: 5),
GYCSRSIPPVCIPD (SEQ ID NO: 6),
GWCSRSYPPVCIPD (SEQ ID NO: 7), and
cyclic peptide with sequence GICSRSLPPICIPD (SEQ ID NO: 3)
and/or their pharmaceutically acceptable salts and esters.

7. The pharmaceutical preparation according to claim 4, characterised by that the peptides are selected from peptides with the following sequences:
GYCSRSYPPVCIPD (SEQ ID NO: 2), and
GICSRSLPPICIPD (SEQ ID NO: 3),
and their pharmaceutically acceptable salts and esters.

8. A kit containing one or more peptides according to general formula (I)
(SEQ ID NO: 20)
GX₁CSX₂SX₃PPX₄CX₅PD (I)
where
X₁ is Y, M, W, I, V, A, and
X₂ is R, K, and
X₃ is Y, F, I, M, L, E, D, H, and
X₄ is V, I, H, and
X₅ is I, V, Y, F, W;
and/or their salt or ester.

9. A procedure for screening compounds potentially inhibiting MASP enzymes, in the course of which
i) a peptide according to general formula (I)
(SEQ ID NO: 20)
GX₁CSX₂SX₃PPX₄CX₅PD (I)
where
X₁ is Y, M, W, I, V, A, and
X₂ is R, K, and
X₃ is Y, F, I, M, L, E, D, H, and
X₄ is V, I, H, and
X₅ is I, V, Y, F, W;
and/or its salt, ester
is added to a solution containing MASP, where the peptide is labeled;
ii) then the solution containing one or more compounds to be tested is added to it;
iii) then the amount of the released marked peptide is measured.

10. The procedure according to claim 9, where the MASP enzyme is selected from MASP-1 or MASP-2 enzyme.

11-14. (canceled)

15. A procedure for isolating MASP enzymes, in the course of which
i) a peptide according to general formula (I)
(SEQ ID NO: 20)
GX₁CSX₂SX₃PPX₄CX₅PD (I)
where
X₁ is Y, M, W, I, V, A, and
X₂ is R, K, and
X₃ is Y, F, I, M, L, E, D, H, and
X₄ is V, I, H, and
X₅ is I, V, Y, F, W;
and/or its salt, ester
is immobilised on a carrier;
ii) the peptide immobilised in this way is contacted with a solution containing MASP enzyme;
iii) the preparation is washed.

16. The procedure according to claim 15, where the MASP enzyme is selected from MASP-1 or MASP-2.

17. The pharmaceutical preparation according to claim 6 and including a matrix providing controlled active agent release.

18. The pharmaceutical preparation according to claim 7 and including a matrix providing controlled active agent release.
